(19) United States
(12) Patent Application Publication
(10) Pub. No.: US 2026/0103688 A1
(43) Pub. Date: Apr. 16, 2026

(54) Constructs and Methods for Biosynthesis of Gastrodin

(71) Applicant: Recombia Biosciences, Inc., Brisbane, CA (US)

(72) Inventors: Michelle Goettge, Lexington, MA (US); Christopher Vickery, Lexington, MA (US); Jing-Ke Weng, Belmont, MA (US)

(21) Appl. No.: 19/126,747

(22) PCT Filed: Nov. 17, 2023
(86) PCT No.: PCT/US2023/080379

§ 371 Date: May 2, 2025

Related U.S. Application Data
(60) Provisional application No. 63/384,169, filed on Nov. 17, 2022.

(51) Int. Cl. C12N 9/10; C12N 15/66; C12P 19/18; C12P 19/44

(52) U.S. Cl. CPC C12N 9/1051; C12N 15/66; C12P 19/18; C12P 19/44; C12N 2800/22; C12Y 204/01017

(57) ABSTRACT

Provided herein are, in various embodiments, host cells, methods, and pharmaceutical compositions including gastrodin, wherein said gastrodin is produced by a transgenic plant or plant cell, fungal cell, yeast cell, insect cell, or bacterial cell. In certain embodiments, the disclosure provides for methods and compositions for the production of gastrodin. In still further embodiments, the disclosure provides for enhanced cells and methods of producing gastrodin.

### RELATED APPLICATION

This application claims the benefit of U.S. Provisional Application No. 63/384,169, filed on Nov. 17, 2022. The entire teachings of the above application are incorporated herein by reference.

### INCORPORATION BY REFERENCE OF MATERIAL IN XML

This application incorporates by reference the Sequence Listing contained in the following extensible Markup Language (XML) file being submitted concurrently herewith:
a) File name: 5767_1006-001_SL.xml; created Nov. 17, 2023, 59,909 Bytes in size.

### BACKGROUND

Gastrodin is a natural product with a range of bioactivities, including neuroprotective, analgesic, and anti-inflammatory effects in both humans and model organisms. Gastrodin is produced by the plant Gastrodia elata, which is also known as Tian Ma in traditional Chinese medicine. Gastrodin is one of the main bioactive components of Gastrodia plant extract. Gastrodin shows efficacy in several pain models and presents itself as a potential treatment for chronic, neuropathic, and chemotherapy-induced pain, both as a single treatment as in combination with other therapeutics.

To achieve the final conversion of 4-hydroxybenzyl alcohol into gastrodin, which requires a glycosyltransferase (UGT) enzyme that utilizes UDP-glucose as a sugar donor. Several enzymes have been described as gastrodin synthases, including UGT73B6 and AsUGT, and have been engineered into heterologous organisms for the production of gastrodin. (CN113755354A; herein incorporated by reference in its entirety).

However, efficient biosynthesis of gastrodin in such heterologous organisms is dependent on the successful transformation of multiple enzymes involved in the glucose to gastrodin biosynthetic pathway. Such processes are cost and time inefficient and result in poor yields. Accordingly, there exists a need for constructs and methods for efficient biosynthesis of gastrodin.

### SUMMARY

In an aspect, the present disclosure provides for a host cell including a transgene encoding a heterologous uridine 5′-diphospho-glucosyltransferase (UGT) operably linked to a promoter, wherein the heterologous UGT includes an amino acid sequence having at least 75% amino acid sequence identity to SEQ ID NO: 2, and wherein the amino acid sequence of the heterologous UGT includes one or more of: M at position 88 of SEQ ID NO: 2; F, Y, or W at position 119 of SEQ ID NO: 2; F, Y, or W at position 145 of SEQ ID NO: 2; L or M at position 149 of SEQ ID NO: 2; M at position 198 of SEQ ID NO: 2; and F at position 383 of SEQ ID NO: 2.

In another aspect, the disclosure provides for a host cell including a transgene encoding a heterologous uridine 5′-diphospho-glucosyltransferase (UGT) operably linked to a promoter, wherein the heterologous UGT includes an amino acid sequence having at least 75% amino acid sequence identity to SEQ ID NO: 28, and wherein the amino acid sequence of the heterologous UGT includes one or more of: M at position 93 of SEQ ID NO: 28; F, Y or W at position 129 of SEQ ID NO: 28; F, Y or W at position 150 of SEQ ID NO: 28; L or M at position 154 of SEQ ID NO: 28; M at position 203 of SEQ ID NO: 28; and F at position 391 of SEQ ID NO: 28.

In another aspect, the disclosure provides for a method of producing gastrodin in a host cell, the method including culturing the host cell in cell culture medium including 4-hydroxybenzyl alcohol, wherein the host cell expresses a transgene that encodes a heterologous uridine 5′-diphospho-glucosyltransferase (UGT) operably linked to a promoter, wherein the heterologous UGT includes an amino acid sequence having at least 75% amino acid sequence identity to SEQ ID NO: 2, and wherein the amino acid sequence of the heterologous UGT includes one or more of: M at position 88 of SEQ ID NO: 2; F, Y, or W at position 119 of SEQ ID NO: 2; F, Y or W at position 145 of SEQ ID NO: 2; L or M at position 149 of SEQ ID NO: 2; M at position 198 of SEQ ID NO: 2; and F at position 383 of SEQ ID NO: 2.

In another aspect, the disclosure provides for a method of producing gastrodin in a host cell, the method including culturing the host cell in cell culture medium including 4-hydroxybenzyl alcohol, wherein the host cell expresses a transgene that encodes a heterologous uridine 5′-diphospho-glucosyltransferase (UGT) operably linked to a promoter, wherein the heterologous UGT includes an amino acid sequence having at least 75% amino acid sequence identity to SEQ ID NO: 28, and wherein the amino acid sequence of the heterologous UGT includes one or more of: M at position 93 of SEQ ID NO: 28; F, Y or W at position 129 of SEQ ID NO: 28; F, Y or W at position 150 of SEQ ID NO: 28; L or M at position 154 of SEQ ID NO: 28; M at position 203 of SEQ ID NO: 28; and F at position 391 of SEQ ID NO: 28.

In another aspect, the disclosure provides for a vector including a nucleic acid encoding a gastrodin synthase for converting 4-hydroxybenzyl alcohol into gastrodin, wherein the gastrodin synthase can have at least about 75% amino acid sequence identity to SEQ ID NO: 2.

In another aspect, the disclosure provides for a vector including a nucleic acid encoding a gastrodin synthase for converting 4-hydroxybenzyl alcohol into gastrodin, wherein the nucleic acid can have at least about 75% amino acid sequence identity to SEQ ID NO: 28.

In another aspect, the disclosure provides for a method of making a transgenic host cell, the method including introducing a vector into a host cell, the vector including a nucleic acid encoding a heterologous uridine 5′-diphospho-glucosyltransferase (UGT) operably linked to a promoter, wherein the heterologous UGT includes an amino acid sequence having at least 75% amino acid sequence identity to SEQ ID NO: 2, and wherein the amino acid sequence of the heterologous UGT includes one or more of: M at position 88 of SEQ ID NO: 2; F, Y or W at position 119 of SEQ ID NO: 2; F, Y or W at position 145 of SEQ ID NO: 2; L or M at position 149 of SEQ ID NO: 2; M at position 198 of SEQ ID NO: 2; and F at position 383 of SEQ ID NO: 2.

In another aspect, the disclosure provides for a method of making a transgenic host cell, the method including introducing a vector into a host cell, the vector including a nucleic acid encoding a heterologous uridine 5′-diphospho-glucosyltransferase (UGT) operably linked to a promoter, wherein the heterologous UGT includes an amino acid sequence having at least 75% amino acid sequence identity to SEQ ID NO: 28, and wherein the amino acid sequence of the heterologous UGT includes one or more of: M at position 93 of SEQ ID NO: 28; F, Y or W at position 129 of SEQ ID NO: 28; F, Y or W at position 150 of SEQ ID NO: 28; L or M at position 154 of SEQ ID NO: 28; M at position 203 of SEQ ID NO: 28; and F at position 391 of SEQ ID NO: 28.

In another aspect, the disclosure provides for a pharmaceutical composition including gastrodin, wherein said gastrodin is produced by a transgenic plant or plant cell, fungal cell, yeast cell, insect cell, or bacterial cell.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows transformation of 4-hydroxybenzyl alcohol into gastrodin. The use of UDP-glucose sugar transferase (UGT) enzyme, GeUGT, to catalyze the transfer of glucose onto the 4-hydroxy group of 4-hydroxybenzylalcohol using UDP-glucose as the glucose donor is described. The reaction produces gastrodin and UDP as a byproduct.

FIG. 2 shows that GeUGT is more efficient than the previously described AsUGT at converting 4-HBA into gastrodin. After 48-hour incubation of 4-HBA with E. coli strains containing either GeUGT or AsUGT, 4-HBA conversion was measured by HPLC analysis. GeUGT exhibited total conversion of 4-HBA to gastrodin, unlike AsUGT, which did not completely convert the 4-HBA in the media. Standards were run in growth media and used to identify both 4-HBA and gastrodin in the tested strains.

FIG. 3 shows a total of 11 UGTs identified as potentially capable of converting 4-HBA into gastrodin. In addition to GeUGT, 11 previously described UGTs were discovered to have gastrodin synthase activity, which is an activity not previously reported for these enzymes. These enzymes were assayed as before with GeUGT, and their activity was compared after 24 h and 48 h.

FIG. 4 shows a sequence alignment of GeUGT (SEQ ID NO; 2), AsUGT (SEQ ID NO: 26), and the 11 additional gastrodin synthase enzymes described (SEQ ID NOs: 4, 6, 8, 10, 12, 14, 16, 18, 20, 22, and 24) as well as a consensus sequence (SEQ ID NO: 28). Sequence analysis reveals that residues M88, F119, I139, F145, L149, M198, and F383 of GeUGT are almost completely unique to this enzyme, and could potentially explain the highly active nature of this enzyme.

FIG. 5A shows an image of the GeUGT active site and identified amino acid residues. FIG. 5B shows an image of the AsUGT active site and identified amino acid residues.

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines, and animals. The present disclosure is not limited by the illustrated ordering of acts or events, as acts may occur in different orders and/or concurrently with other acts or events. Furthermore, not all illustrated acts, steps, or events are required to implement a methodology in accordance with the present disclosure. Many of the techniques and procedures described, or referenced herein, are well understood and commonly employed using conventional methodology by those skilled in the art.

Unless otherwise defined, all terms of art, notations, and other scientific terms or terminology used herein are intended to have the meanings commonly understood by those of skill in the art to which this disclosure pertains. In various cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an,” and “the” should be understood to include plural reference unless the context clearly indicates otherwise.

Throughout this specification and the claims which follow, unless the context requires otherwise, the word “comprise,” and variations such as “comprises” and “comprising,” will be understood to imply the inclusion of, e.g., a stated integer or step or group of integers or steps, but not the exclusion of any other integer or step or group of integers or steps. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in various embodiments, be replaced with the term “consisting of,” or “consisting essentially of” to vary the scope of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

### Nucleic Acids

As used herein, the term “nucleic acid” refers to a polymer including multiple nucleotide monomers (e.g., ribonucleotide monomers or deoxyribonucleotide monomers). “Nucleic acid” includes, for example, DNA (e.g., genomic DNA and cDNA), RNA, and DNA-RNA hybrid molecules. Nucleic acid molecules can be naturally occurring, recombinant, or synthetic. In addition, nucleic acid molecules can be single-stranded, double-stranded or triple-stranded. In certain embodiments, nucleic acid molecules can be modified. In the case of a double-stranded polymer, “nucleic acid” can refer to either or both strands of the molecule.

The terms “nucleotide” and “nucleotide monomer” refer to naturally occurring ribonucleotide or deoxyribonucleotide monomers, as well as non-naturally occurring derivatives and analogs thereof. Accordingly, nucleotides can include, for example, naturally occurring bases (e.g., adenosine, thymidine, guanosine, cytidine, uridine, inosine, deoxyadenosine, deoxythymidine, deoxyguanosine, or deoxycytidine) and nucleotides including modified bases known in the art.

As used herein, “wildtype” refers to the canonical amino acid sequence as found in nature. As those of skill in the art would appreciate, a nucleic acid sequence can be modified, (e.g., for codon optimization in a host cell (e.g., bacteria, yeast, and plant host cells)).

As used herein, the term “sequence identity,” refers to the extent to which two nucleotide sequences, or two amino acid sequences, have the same residues at the same positions when the sequences are aligned to achieve a maximal level of identity, expressed as a percentage. For sequence alignment and comparison, typically one sequence is designated as a reference sequence, to which a test sequences are compared. The sequence identity between reference and test sequences is expressed as the percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide or amino acid residue at 70% of the same positions over the entire length of the reference sequence.

Alignment of sequences for comparison to achieve maximal levels of identity can be readily performed by a person of ordinary skill in the art using an appropriate alignment method or algorithm. In some instances, the alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. A commonly used tool for determining percent sequence identity is Protein Basic Local Alignment Search Tool (BLASTP) available through National Center for Biotechnology Information, National Library of Medicine, of the United States National Institutes of Health. (Altschul et al., 1990).

In various embodiments, two nucleotide sequences, or two amino acid sequences, can have at least, e.g., 70%, 75%, 80%, 85%, 86%, 87%, 88%, 89%, 90%, 91%, 92%, 93%, 94%, 95%, 96%, 97%, 98%, 99%, or more, sequence identity. When ascertaining percent sequence identity to one or more sequences described herein, the sequences described herein are the reference sequences.

Various embodiments of the invention relate to a nucleic acid coding sequence (e.g., dsDNA, cDNA) encoding one or more of the enzymes described herein, including those nucleic acid sequences provided in SEQ ID NO: 1 and SEQ ID NO: 27. Many different nucleic acids can encode a UGT of the disclosure due to the degeneracy of the genetic code. Nucleic acids can also differ, for example, as a result of one or more substitutions (e.g., silent substitutions).

### Enzymes

As used herein, the term 5′-diphospho-glucosyltransferase (UGT) refers to an enzyme that catalyzes conversion of 4-hydroxybenzyl alcohol into gastrodin. Methods and assays for determining whether an enzyme catalyzes conversion of 4-hydroxybenzyl alcohol to gastrodin are known in the art, and include enzyme activity assays and liquid chromatography to assess retention time of metabolites. Chemical structure can also be assessed by nuclear magnetic resonance (NMR) or liquid chromatography-mass spectrometry. An example of a UGT is SEQ ID NO: 2, which is the amino acid sequence of a UGT identified in Gastrodia elata (GeUGT).

Aspects of the disclosure provide for a UGT with at least about 70% or more sequence identity to SEQ ID NO: 2, or a biologically active fragment thereof. In other aspects, the disclosure provides a UGT with at least about 75% or more sequence identity to SEQ ID NO: 2, or a biologically active fragment thereof. The disclosure provides for a UGT with at least about 76% or more sequence identity to SEQ ID NO: 2, or a biologically active fragment thereof. In still further aspects, the disclosure provides for a UGT with at least about 77% or more sequence identity to SEQ ID NO: 2, or a biologically active fragment thereof. Other aspects of the disclosure provide for a UGT with at least about 78% or more sequence identity to SEQ ID NO: 2, or a biologically active fragment thereof. In other aspects, the disclosure provides for a UGT with at least about 78% or more sequence identity to SEQ ID NO: 2, or a biologically active fragment thereof. In still further embodiments, the disclosure provides for a UGT with at least about 79% or more sequence identity to SEQ ID NO: 2, or a biologically active fragment thereof. In still further aspects, the disclosure provides for a UGT with at least about 80% or more sequence identity to SEQ ID NO: 2, or a biologically active fragment thereof. In still further aspects, the disclosure provides for a UGT with at least about 81% or more sequence identity to SEQ ID NO: 2, or a biologically active fragment thereof. In still other embodiments, the disclosure provides for a UGT with at least about 82% or more sequence identity to SEQ ID NO: 2, or a biologically active fragment thereof. In other embodiments, the disclosure provides for a UGT with at least about 83% or more sequence identity to SEQ ID NO: 2, or a biologically active fragment thereof. In still other embodiments, the disclosure provides for a UGT with at least about 84% or more sequence identify to SEQ ID NO: 2, or a biologically active fragment thereof. In further embodiments, the disclosure provides for a UGT with at least about 85% or more sequence identity to SEQ ID NO: 2, or a biologically active fragment thereof. In other aspects, the disclosure provides for a UGT with at least about 86% or more sequence identity to SEQ ID NO: 2, or a biologically active fragment thereof. In still other aspects, the disclosure provides for a UGT with at least about 87% or more sequence identify to SEQ ID NO: 2, or a biologically active fragment thereof. In other aspects, the disclosure provides for a UGT with at least about 88% or more sequence identify to SEQ ID NO: 2, or a biologically active fragment thereof. In further embodiments, the disclosure provides for a UGT with at least about 89% or more sequence identity to SEQ ID NO: 2, or a biologically active fragment thereof. The disclosure also provides for a UGT with at least about 90% or more sequence identity to SEQ ID NO: 2, or a biologically active fragment thereof. In further aspects, the disclosure provides for a UGT with at least about 91% or more sequence identity to SEQ ID NO: 2, or a biologically active fragment thereof. In still further embodiments, the disclosure provides for a UGT with at least about 92% or more sequence identity to SEQ ID NO: 2, or a biologically active fragment thereof. In aspects, the disclosure provides for a UGT with at least about 93% or more sequence identity to SEQ ID NO: 2, or a biologically active fragment thereof. In other embodiments, the disclosure provides for a UGT with at least about 94% or more sequence identity to SEQ ID NO: 2, or a biologically active fragment thereof. In further embodiments, disclosure also provides for a UGT with at least about 95% or more sequence identity to SEQ ID NO: 2, or a biologically active fragment thereof. In embodiments, the disclosure provides for a UGT with at least about 96% or more sequence identity to SEQ ID NO: 2, or a biologically active fragment thereof. Still further, aspects of the disclosure provide for a UGT with at least about 97% or more sequence identity to SEQ ID NO: 2, or a biologically active fragment thereof. In other embodiments, the disclosure provides for a UGT with at least about 98% or more sequence identity to SEQ ID NO: 2, or a biologically active fragment thereof. In still further embodiments, the disclosure provides for a UGT with at least about 99% or more sequence identity to SEQ ID NO: 2, or a biologically active fragment thereof. The disclosure also provides a UGT sharing sequence identity with SEQ ID NO: 2, or a biologically active fragment thereof.

In one aspect, the present disclosure provides a heterologous UGT operably linked to a promoter, wherein the heterologous UGT includes an amino acid sequence having at least 75% amino acid sequence identity to SEQ ID NO: 2, and wherein the amino acid sequence of the heterologous UGT includes one or more of: M at position 88 of SEQ ID NO: 2; F, Y, or W at position 119 of SEQ ID NO: 2; F, Y, or W at position 145 of SEQ ID NO: 2; L or M at position 149 of SEQ ID NO: 2; M at position 198 of SEQ ID NO: 2; and F at position 383 of SEQ ID NO: 2. In embodiments, the heterologous UGT includes at least two of: M at position 88 of SEQ ID NO: 2; F, Y, or W at position 119 of SEQ ID NO: 2; F, Y, or W at position 145 of SEQ ID NO: 2; L or M at position 149 of SEQ ID NO: 2; M at position 198 of SEQ ID NO: 2; or F at position 383 of SEQ ID NO: 2. As a non-limiting example, a heterologous UGT can include M at position 88 of SEQ ID NO: 2 and F at position 119 of SEQ ID NO: 2. Embodiments of the disclosure provide for a heterologous UGT includes at least three of: M at position 88 of SEQ ID NO: 2; F, Y, or W at position 119 of SEQ ID NO: 2; F, Y, or W at position 145 of SEQ ID NO: 2; L or M at position 149 of SEQ ID NO: 2; M at position 198 of SEQ ID NO: 2; or F at position 383 of SEQ ID NO: 2. As a non-limiting example, a heterologous UGT can include M at position 88 of SEQ ID NO: 2; F at position 119 of SEQ ID NO: 2; and F at position 145 of SEQ ID NO: 2. In embodiments, the heterologous UGT includes at least four of: M at position 88 of SEQ ID NO: 2; F, Y, or W at position 119 of SEQ ID NO: 2; F, Y, or W at position 145 of SEQ ID NO: 2; L or M at position 149 of SEQ ID NO: 2; M at position 198 of SEQ ID NO: 2; or F at position 383 of SEQ ID NO: 2. As a non-limiting example, a heterologous UGT can include M at position 88 of SEQ ID NO: 2; F at position 119 of SEQ ID NO: 2; F at position 145 of SEQ ID NO: 2; and F at position 383 of SEQ ID NO: 2. In other embodiments, the heterologous UGT includes at least five of: M at position 88 of SEQ ID NO: 2; F, Y, or W at position 119 of SEQ ID NO: 2; F, Y, or W at position 145 of SEQ ID NO: 2; L or M at position 149 of SEQ ID NO: 2; M at position 198 of SEQ ID NO: 2; or F at position 383 of SEQ ID NO: 2. As a non-limiting example, a heterologous UGT can include M at position 88 of SEQ ID NO: 2; F at position 119 of SEQ ID NO: 2; F at position 145 of SEQ ID NO: 2; L at position 149 of SEQ ID NO: 2; and F at position 383 of SEQ ID NO: 2. In still further embodiments, the heterologous UGT includes all of: M at position 88 of SEQ ID NO: 2; F, Y, or W at position 119 of SEQ ID NO: 2; F, Y, or W at position 145 of SEQ ID NO: 2; L or M at position 149 of SEQ ID NO: 2; M at position 198 of SEQ ID NO: 2; or F at position 383 of SEQ ID NO: 2. As a non-limiting example, a heterologous UGT can include M at position 88 of SEQ ID NO: 2; F at position 119 of SEQ ID NO: 2; F at position 145 of SEQ ID NO: 2; L at position 149 of SEQ ID NO: 2; M at position 198 of SEQ ID NO: 2; and F at position 383 of SEQ ID NO: 2.

In still further embodiments, the disclosure provides a UGT operably linked to a promoter, wherein the UGT included an amino acid sequence, wherein the amino acid sequence does not have one or more of the following residues: I at position 88 of SEQ ID NO: 2; L at position 119 of SEQ ID NO: 2; C at position 145 of SEQ ID NO: 2; F at position 149 of SEQ ID NO: 2; L at position 198 of SEQ ID NO: 2; or Y at position 383 of SEQ ID NO: 2.

In any of the foregoing embodiments, the disclosure provide for a UGT with at least about 70% or more sequence identity to SEQ ID NO: 28, or a biologically active fragment thereof. In other aspects, the disclosure provides a UGT with at least about 75% or more sequence identity to SEQ ID NO: 28, or a biologically active fragment thereof. In aspects, the disclosure provides for a UGT with at least about 76% or more sequence identity to SEQ ID NO: 28, or a biologically active fragment thereof. In still further aspects, the disclosure provides for a UGT with at least about 77% or more sequence identity to SEQ ID NO: 28, or a biologically active fragment thereof. Other aspects of the disclosure provide for a UGT with at least about 78% or more sequence identity to SEQ ID NO: 28, or a biologically active fragment thereof. In other aspects, the disclosure provides for a UGT with at least about 78% or more sequence identity to SEQ ID NO: 28, or a biologically active fragment thereof. In still further embodiments, the disclosure provides for a UGT with at least about 79% or more sequence identity to SEQ ID NO: 28, or a biologically active fragment thereof. In still further aspects, the disclosure provides for a UGT with at least about 80% or more sequence identity to SEQ ID NO: 28, or a biologically active fragment thereof. In still further aspects, the disclosure provides for a UGT with at least about 81% or more sequence identity to SEQ ID NO: 28, or a biologically active fragment thereof. In still other embodiments, the disclosure provides for a UGT with at least about 82% or more sequence identity to SEQ ID NO: 28, or a biologically active fragment thereof. In other embodiments, the disclosure provides for a UGT with at least about 83% or more sequence identity to SEQ ID NO: 28, or a biologically active fragment thereof. In still other embodiments, the disclosure provides for a UGT with at least about 84% or more sequence identify to SEQ ID NO: 28, or a biologically active fragment thereof. In further embodiments, the disclosure provides for a UGT with at least about 85% or more sequence identity to SEQ ID NO: 28, or a biologically active fragment thereof. In other aspects, the disclosure provides for a UGT with at least about 86% or more sequence identity to SEQ ID NO: 28, or a biologically active fragment thereof. In still other aspects, the disclosure provides for a UGT with at least about 87% or more sequence identify to SEQ ID NO: 28, or a biologically active fragment thereof. In other aspects, the disclosure provides for a UGT with at least about 88% or more sequence identify to SEQ ID NO: 28, or a biologically active fragment thereof. In further embodiments, the disclosure provides for a UGT with at least about 89% or more sequence identity to SEQ ID NO: 28, or a biologically active fragment thereof. The disclosure also provides for a UGT with at least about 90% or more sequence identity to SEQ ID NO: 28, or a biologically active fragment thereof. In further aspects, the disclosure provides for a UGT with at least about 91% or more sequence identity to SEQ ID NO: 28, or a biologically active fragment thereof. In still further embodiments, the disclosure provides for a UGT with at least about 92% or more sequence identity to SEQ ID NO: 28, or a biologically active fragment thereof. In aspects, the disclosure provides for a UGT with at least about 93% or more sequence identity to SEQ ID NO: 28, or a biologically active fragment thereof. In other embodiments, the disclosure provides for a UGT with at least about 94% or more sequence identity to SEQ ID NO: 28, or a biologically active fragment thereof. In further embodiments, disclosure also provides for a UGT with at least about 95% or more sequence identity to SEQ ID NO: 28, or a biologically active fragment thereof. In embodiments, the disclosure provides for a UGT with at least about 96% or more sequence identity to SEQ ID NO: 28, or a biologically active fragment thereof. Still further, aspects of the disclosure provide for a UGT with at least about 97% or more sequence identity to SEQ ID NO: 28, or a biologically active fragment thereof. In other embodiments, the disclosure provides for a UGT with at least about 98% or more sequence identity to SEQ ID NO: 28, or a biologically active fragment thereof. In still further embodiments, the disclosure provides for a UGT with at least about 99% or more sequence identity to SEQ ID NO: 28, or a biologically active fragment thereof. The disclosure also provides a UGT sharing sequence identity with SEQ ID NO: 28, or a biologically active fragment thereof.

In one aspect, the present disclosure provides a heterologous UGT operably linked to a promoter, wherein the heterologous UGT includes an amino acid sequence having at least about 75% amino acid sequence identity to SEQ ID NO: 28, and wherein the amino acid sequence of the heterologous UGT includes one or more of: M at position 93 of SEQ ID NO: 28; F, Y or W at position 129 of SEQ ID NO: 28; F, Y or W at position 150 of SEQ ID NO: 28; L or M at position 154 of SEQ ID NO: 28; M at position 203 of SEQ ID NO: 28; and F at position 391 of SEQ ID NO: 28.

### Vectors

The terms “vector”, “vector construct” and “expression vector” mean the vehicle by which a DNA or RNA sequence (e.g., a foreign gene) can be introduced into a host cell, so as to transform the host and promote expression (e.g., transcription and translation) of the introduced sequence. Vectors typically include the DNA of a transmissible agent, into which foreign DNA encoding a protein is inserted by restriction enzyme technology. A common type of vector is a “plasmid”, which generally is a self-contained molecule of double-stranded DNA that can readily accept additional (foreign) DNA and which can readily introduced into a suitable host cell. A large number of vectors, including plasmid and fungal vectors, have been described for replication and/or expression in a variety of eukaryotic and prokaryotic hosts.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself (e.g., the resulting protein) may also be said to be “expressed” by the cell. A polynucleotide or polypeptide is expressed recombinantly, for example, when it is expressed or produced in a foreign host cell under the control of a foreign or native promoter, or in a native host cell under the control of a foreign promoter.

Gene delivery vectors generally include a transgene (e.g., nucleic acid encoding an enzyme) operably linked to a promoter and other nucleic acid elements required for expression of the transgene in the host cells into which the vector is introduced. Suitable promoters for gene expression and delivery constructs are known in the art. For bacterial host cells, suitable promoters, include, but are not limited to promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xy1A and xy1B genes, and prokaryotic beta-lactamase gene (See e.g., Villa-Kamaroff et al., Proc. Natl. Acad. Sci. USA 75:3727-3731, 1978), as well as the tac promoter (See e.g., DeBoer et al., Proc. Natl. Acad. Sci. USA 80:21-25, 1983). Examples of promoters for filamentous fungal host cells, include, but are not limited to promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Examples of yeast cell promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (See e.g., Romanos et al., Yeast 8:423-488, 1992). The selection of a suitable promoter is within the skill in the art. The recombinant plasmids can also include inducible, or regulatable, promoters for expression of an enzyme in cells.

Various gene delivery vehicles are known in the art and include both viral and non-viral (e.g., naked DNA, plasmid) vectors. Viral vectors suitable for gene delivery are known to those skilled in the art. Such viral vectors, include, but are not limited to vectors derived from the herpes virus, baculovirus vectors, lentiviral vectors, retroviral vectors, adenoviral vectors and adeno-associated viral vectors (AAVs). Vectors derived from plant viruses can also be used, such as the viral backbones of the RNA viruses Tobacco mosaic virus (TMV), Potato virus X (PVX) and Cowpea mosaic virus (CPMV), and the DNA geminivirus Bean yellow dwarf virus. The viral vector can be replicating or non-replicating

Non-viral vectors include naked DNA and plasmids, among others. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and such vectors may be introduced into many appropriate host cells, using methods disclosed or cited herein or otherwise known to those skilled in the relevant art.

In certain embodiments, the vector includes a transgene operably linked to a promoter. The transgene encodes a biologically active molecule, such as an enzyme (e.g., a heterologous UGT) described herein.

To facilitate the introduction of the gene delivery vector into host cells, the vector can be combined with different chemical means such as colloidal dispersion systems (e.g., a macromolecular complex, nanocapsules, microspheres, beads) or lipid-based systems (e.g., oil-in-water emulsions, micelles, liposomes).

The disclosure also provides for embodiments relating to a vector including a nucleic acid encoding an enzyme described herein. In certain embodiments, the vector is a plasmid, and includes any one or more plasmid sequences (e.g., a promoter sequence, a selection marker sequence, and/or a locus-targeting sequence).

Although the genetic code is degenerate in that most amino acids are represented by multiple codons (called “synonyms” or “synonymous” codons), it is understood in the art that codon usage by particular organisms is nonrandom and biased towards particular codon triplets. Accordingly, in various embodiments, the vector includes a nucleotide sequence that can be optimized for expression in a particular type of host cell (e.g., through codon optimization). Codon optimization refers to a process in which a polynucleotide encoding a protein of interest is modified to replace particular codons in that polynucleotide with codons that encode the same amino acid(s) but are more commonly used/recognized in the host cell in which the nucleic acid is being expressed. In various aspects, the polynucleotides described herein are codon optimized for expression in a bacterial cell (e.g., E. coli) or a yeast cell (e.g., S. cerevisiae).

### Host Cells

A wide variety of host cells can be used, including fungal cells, bacterial cells, plant cells, insect cells, and mammalian cells.

In embodiments of the disclosure, the host cell is a fungal cell, such as a yeast cell and an Aspergillus spp cell. A wide variety of yeast cells are suitable, such as cells of the genus Pichia, including Pichia pastoris and Pichia stipitis; cells of the genus Saccharomyces, including Saccharomyces cerevisiae; cells of the genus Schizosaccharomyces, including Schizosaccharomyces pombe; and cells of the genus Candida, including Candida albicans.

In other embodiments, the host cell is a bacterial cell. A wide variety of bacterial cells are suitable, such as cells of the genus Escherichia, including Escherichia coli; cells of the genus Bacillus, including Bacillus subtilis; cells of the genus Pseudomonas, including Pseudomonas aeruginosa; and cells of the genus Streptomyces, including Streptomyces griseus.

In other embodiments, the host cell is a plant cell. A wide variety of cells from a plant are suitable, including cells from a Nicotiana benthamiana plant. In other embodiments, the plant belongs to a genus selected from the group consisting of Arabidopsis, Beta, Glycine, Helianthus, Solanum, Triticum, Oryza, Brassica, Medicago, Prunus, Malus, Hordeum, Musa, Phaseolus, Citrus, Piper, Sorghum, Daucus, Manihot, Capsicum, and Zea.

In still other embodiments, the host cell is an insect cell, such as a Spodoptera frugiperda cell, such as Spodoptera frugiperda Sf9 cell line and Spodoptera frugiperda Sf21 In further embodiments, the host cell is a mammalian cell.

In further embodiments, the host cell is an Escherichia coli cell. In embodiments of the disclosure, the host cell is a Nicotiana benthamiana cell. In other embodiments, the cell is a Saccharomyces cerevisiae cell.

As used herein, the term “host cell” encompasses cells in cell culture and also cells within an organism (e.g., a plant).

Various embodiments relate to a host cell including a vector as described herein. In certain embodiments, the host cell is an Escherichia coli cell, a Nicotiana benthamiana cell, or a Saccharomyces cerevisiae cell.

In embodiments, the hosts cells are cultured in a cell culture medium, such as a standard cell culture medium known in the art to be suitable for the particular host cell.

In other aspects, the disclosure provides for a method of producing gastrodin in a host cell, including culturing the host cell in cell culture medium including 4-hydroxybenzyl alcohol, wherein the host cell expresses a transgene that encodes a heterologous uridine 5′-diphospho-glucosyltransferase (UGT) operably linked to a promoter,

In embodiments of the disclosure, the host cell is a plant cell, a fungal cell, a yeast cell, an insect cell, or a bacterial cell. In still further aspects, the disclosure provides for a heterologous UGT is codon-optimized for expression in the host cell.

In aspects of the disclosure, the method provides for a cell culture medium further including glucose. In other aspects, the disclosure provides for a method including making the host cell, the method including introducing a vector into the host cell, the vector including a nucleic acid encoding the heterologous uridine 5′-diphospho-glucosyltransferase (UGT) operably linked to the promoter.

In still further aspects, the disclosure provides for a method wherein the gastrodin is extracted using maceration, percolation, decoction, reflux extraction, soxhlet extraction, pressurized liquid extraction, supercritical fluid extraction, ultrasound assisted extraction, pulsed electric field extraction, enzyme assisted extraction, hydro distillation, steam distillation, or any combination thereof.

In further aspects of the disclosure, the method provided herein includes a concentration of gastrodin within the cell culture medium after 24 h incubation wherein the concentration is at least about 4 mM, 5 mM, 6 mM, 7 mM or 8 mM. In other aspects, the disclosure provides for a concentration of 4-hydroxybenzyl alcohol within the cell culture medium after 24 hr incubation wherein the concentration is not greater than 2 mM, 1.5 mM, or 1 mM.

### Methods of Making Transgenic Host Cells

Described herein are methods of making a transgenic host cell. The transgenic host cells can be made, for example, by introducing one or more of the vector embodiments described herein into the host cell.

In further embodiments, the disclosure provides for a method of making a transgenic host cell, the method including introducing a vector into a host cell, the vector including a nucleic acid encoding a heterologous uridine 5′-diphospho-glucosyltransferase (UGT) operably linked to a promoter.

In other embodiments, one or more of the nucleic acids are integrated into the genome of the host cell. In still other embodiments, the nucleic acids to be integrated into a host genome can be introduced into the host cell using any of a variety of suitable methodologies known in the art, including, for example, CRISPR-based systems (e.g., CRISPR/Cas9; CRISPR/Cpf1), TALEN systems and Agrobacterium-mediated transformation. However, as those skilled in the art would recognize, transient transformation techniques can be used that do not require integration into the genome of the host cell. In embodiments of the disclosure, nucleic acid (e.g., plasmids) can be introduced that are maintained as episomes, which need not be integrated into the host cell genome.

In certain embodiments, the nucleic acid is introduced into a tissue, cell, or seed of a plant cell. Various methods of introducing nucleic acid into the tissue, cell, or seed of plants are known to one of ordinary skill in the art, such as protoplast transformation. The particular method can be selected based on several considerations, such as, e.g., the type of plant used. For example, the floral dip method, as described herein, is a suitable method for introducing genetic material into a plant. In certain embodiments, the nucleic acid can be delivered into the plant by an Agrobacterium.

### Methods of Making Gastrodin

Described herein are methods of making gastrodin. In various embodiments, the disclosure provides for a pharmaceutical composition consisting of gastrodin, wherein said gastrodin is produced by a transgenic plant or plant cell, fungal cell, yeast cell, insect cell, or bacterial cell.

### Values and Ranges

Unless otherwise indicated or otherwise evident from the context and understanding of one of ordinary skill in the art, values that are expressed as ranges can assume any specific value or subrange within the stated ranges in various embodiments, unless the context clearly dictates otherwise. “About” in reference to a numerical value generally refers to a range of values that fall within +8%, in some embodiments+6%, in some embodiments+4%, in some embodiments+2%, in some embodiments+1%, in some embodiments+0.5% of the value unless otherwise stated or otherwise evident from the context.

### Sequences

Table 1 is a summary of the nucleotide and amino acid sequences disclosed in the sequence listing incorporated herein.

| Enzyme | Nucleotide Sequence | Amino Acid Sequence |
|---|---|---|
| GeUGT | SEQ ID NO: 1 | SEQ ID NO: 2 |
| SiUGT | SEQ ID NO: 3 | SEQ ID NO: 4 |
| CaUGT | SEQ ID NO: 5 | SEQ ID NO: 6 |
| PpUGT | SEQ ID NO: 7 | SEQ ID NO: 8 |
| NbUGT1 | SEQ ID NO: 9 | SEQ ID NO: 10 |
| NbUGT2 | SEQ ID NO: 11 | SEQ ID NO: 12 |
| PcUGT | SEQ ID NO: 13 | SEQ ID NO: 14 |
| WsUGT | SEQ ID NO: 15 | SEQ ID NO: 16 |
| AtUGT1 | SEQ ID NO: 17 | SEQ ID NO: 18 |
| AtUGT2 | SEQ ID NO: 19 | SEQ ID NO: 20 |
| PtUGT | SEQ ID NO: 21 | SEQ ID NO: 22 |
| RrUGT | SEQ ID NO: 23 | SEQ ID NO: 24 |
| AsUGT | SEQ ID NO: 25 | SEQ ID NO: 26 |
| Consensus sequence | SEQ ID NO: 27 | SEQ ID NO: 28 |

### EXEMPLIFICATION

### Example 1

### Materials and Methods

Transcriptomics of G. elata:

To discover enzymes that could convert 4-hydroxybenzyl alcohol to gastrodin, a raw transcriptome of G. elata was processed as previously described.10,11 A transcriptome constructed from this data was searched using NCBI BLAST with AsUGT as a search query. From this transcriptome, 5 candidate genes were chosen as likely UGT enzymes.

Cloning into Expression Vector:

Each putative UGT enzyme was cloned into a pCL1921-derived plasmid, which contained a constitutive pL promoter to drive expression, and a spectinomycin resistance cassette. The UGTs were assembled using Gibson assembly into a pCL1921 backbone and transformed into DH5-a cells and plated on spectinomycin. Sequenced plasmids Plasmids containing UGTs were electroporated into wildtype E. coli C and selected on media containing spectinomycin.

### Testing Strains for Gastrodin Production:

Three colonies from each plasmid transformation were innoculated into 1 mL LB media that contained 10 mM 4-hydroxybenzyl alcohol and 2% glucose. These strains were grown for 48 hours at 37° C. with shaking at 1000 rpm in a deep well plate. Samples were taken at both 24 and 48 hours for analysis.

### Detection and Quantification of Gastrodin:

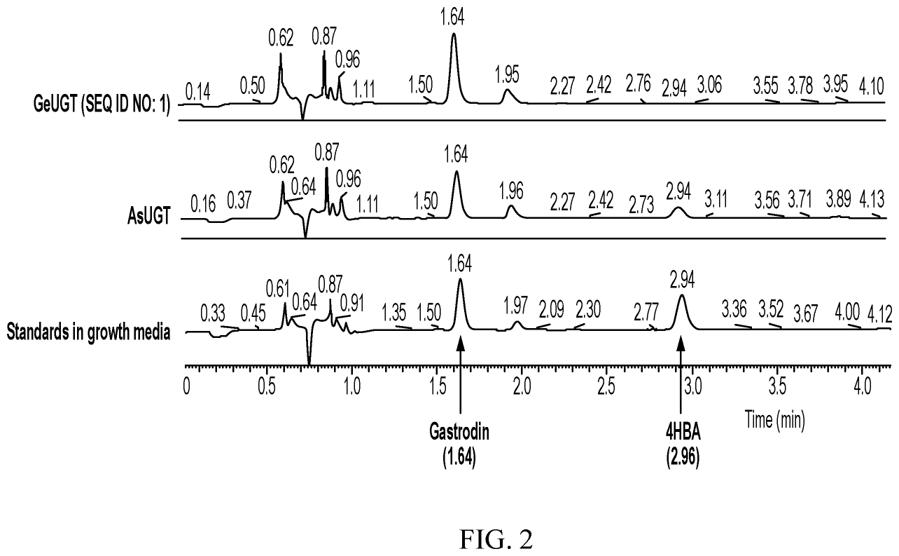

Standards of both gastrodin and 4-hydroxybenzyl alcohol were used to develop an HPLC-based method for detection of these two compounds in the strain testing experiments. At 24 and 48 hours after inoculation, each strain was sampled, centrifuged to remove cells, and the supernatant was removed for HPLC analysis. Strains with gastrodin synthase activity exhibited an appearance of a peak corresponding to gastrodin, while the signal for 4-hydroxybenzyl alcohol decreased. After 48 hours, the conversion of 4-hydroxybenzyl alcohol to gastrodin was assessed (FIG. 2).

### Selection of Other UGTs:

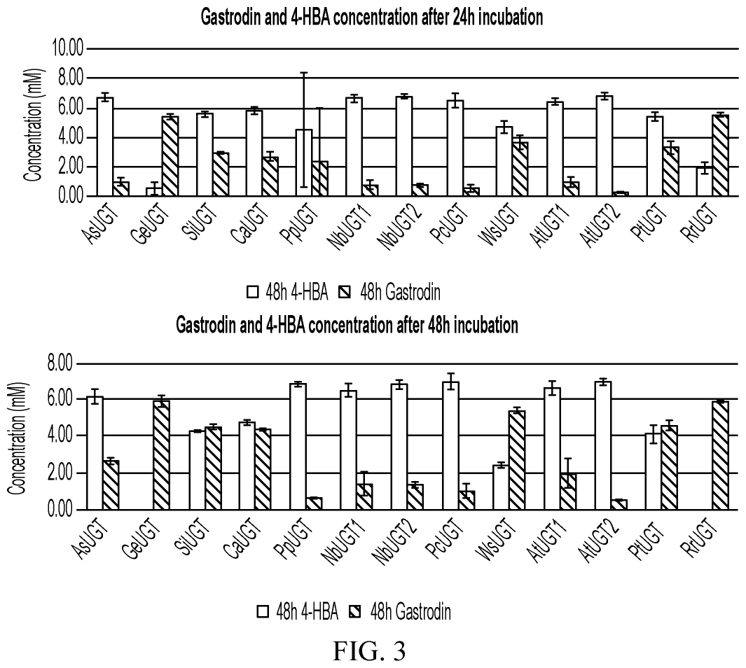

Other similar UGTs, which were predicted to have gastrodin synthase activity based on their sequence, were tested to determine if they could convert 4-hydroxybenzyl alcohol to gastrodin. Through a combination of screening enzymes and searching the NCBI database with a BLAST search, 11 additional UGTs that were able to convert 4-hydroxybenzyl alcohol into gastrodin when assayed as described above (FIG. 3) were identified. No description of gastrodin production by any of these 11 UGTs has been identified. These genes were SiUGT (SEQ ID NO: 4), CaUGT (SEQ ID NO: 6), PpUGT (SEQ ID NO: 8), NbUGT1 (SEQ ID NO: 10), NbUGT2 (SEQ ID NO: 12), PcUGT (SEQ ID NO: 14), WsUGT (SEQ ID NO: 16), AtUGT1 (SEQ ID NO: 18), AtUGT2 (SEQ ID NO: 20), PtUGT (SEQ ID NO: 22), and RrUGT (SEQ ID NO: 24).

### Structural Analysis of AsUGT and Comparison of Active Site Residues:

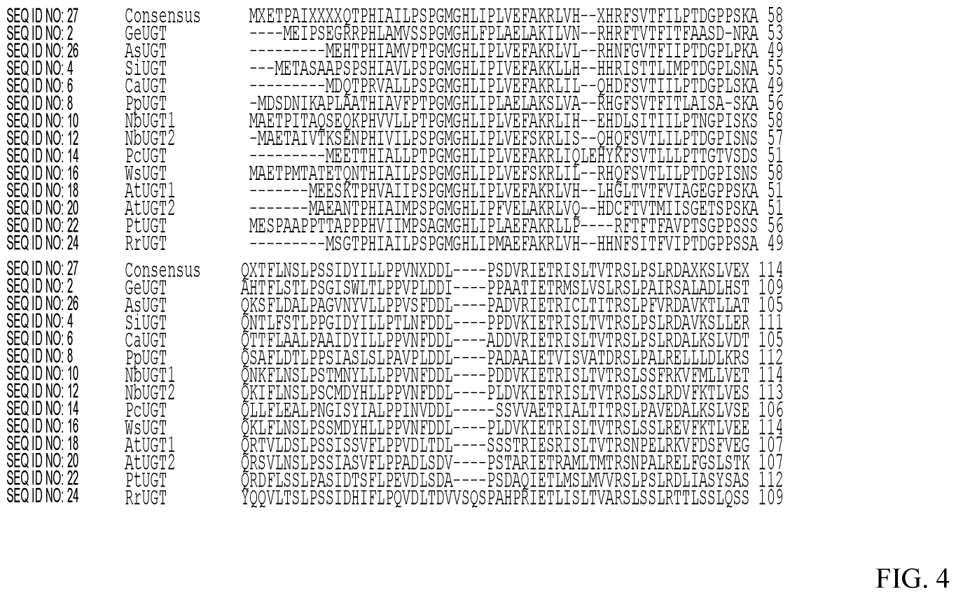

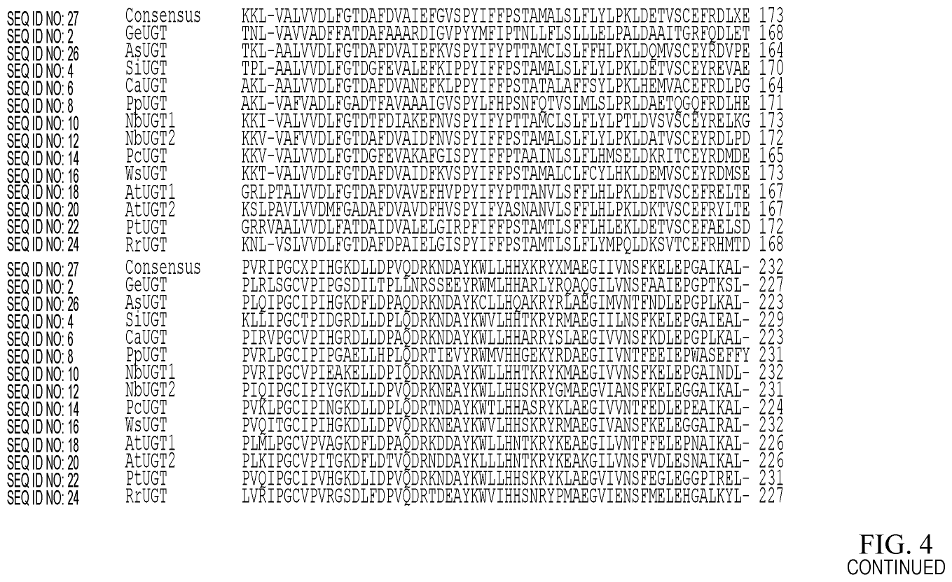

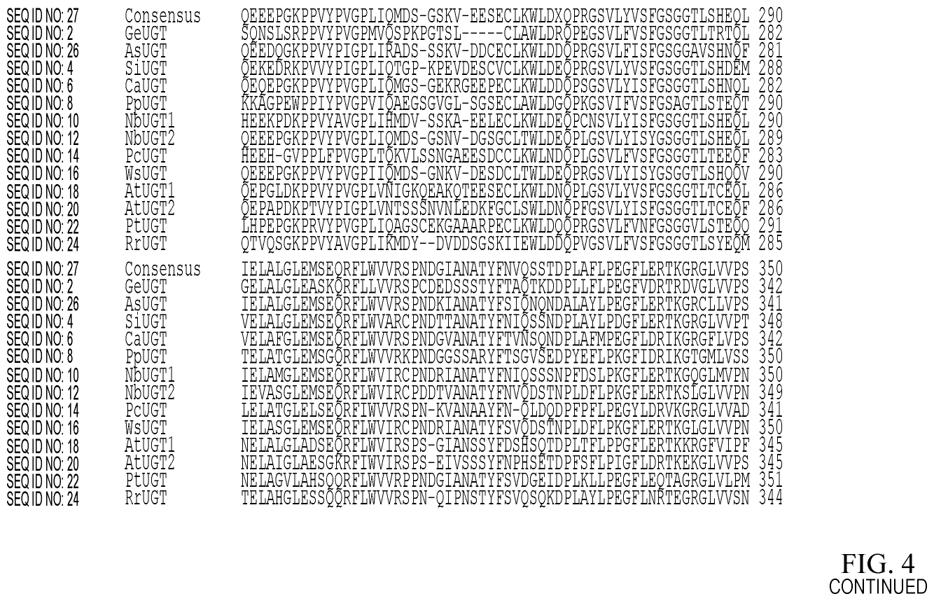

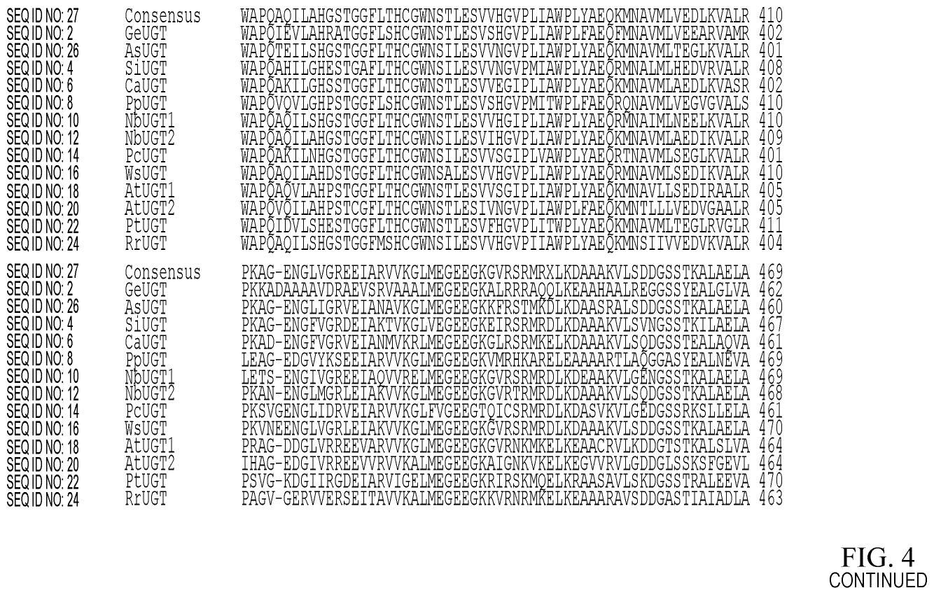

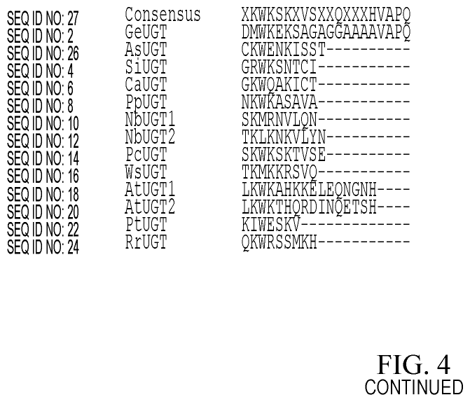

To better understand the residues that contribute to substrate binding and higher activity in GeUGT, a structural model of AsUGT was investigated. After comparing the sequences of GeUGT (SEQ ID NO: 2) and AsUGT (SEQ ID NO: 26), the GeUGT active site residues M88, F119, I139, F145, L149, M198, and F383, which correspond to 184, L115, Y135, C141, F145, L194, and Y382, respectively, in AsUGT were identified. These residues were also found to be unique to GeUGT when compared to the other identified gastrodin synthase enzymes described here (FIG. 4). These active site differences are directly surrounding the putative binding site for 4-hydroxybenzyl alcohol, and may increase the binding affinity of GeUGT for 4-hydroxybenzyl alcohol to lead to the observed enhanced gastrodin synthesis rate of GeUGT.

### Fermentation Conditions:

To investigate the scalability of gastrodin production, E. coli C transformed with a plasmid bearing GeUGT under a pL promoter was cultured in a 1 L bioreactor. The culture was fed glucose and 4-hydroxybenzyl alcohol over a course of 86 hours. A total of 38 g of 4-hydroxybenzyl alcohol was fed to the culture, resulting in a final gastrodin titer of 48.8 g/L.

### Consensus Sequence:

Consensus nucleotide sequence was calculated using all nucleotide sequences and using the MUSCLE alignment algorithm.

### Results and Discussion

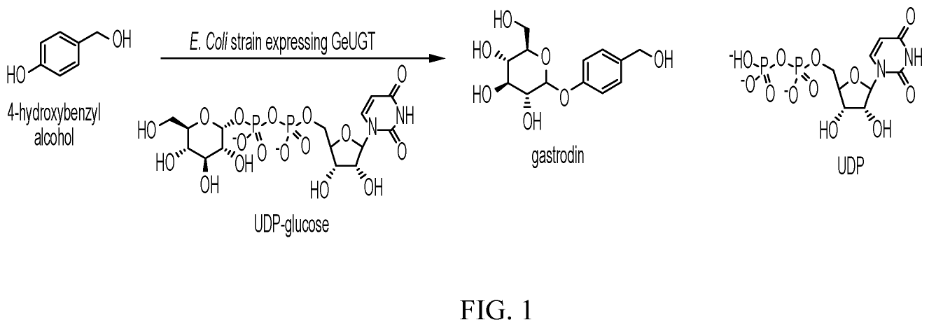

Although gastrodin originates from the plant Gastrodia elata, no enzyme from this plant has been described, thus potential enzymes from this plant by analysis of existing transcriptome data were investigated. The transcriptome analysis of G. elata enabled searching for UGTs that were similar to the known gastrodin synthase AsUGT. Herein, the present disclosure describes the use of the native Gastrodia UGT enzyme (GeUGT, SEQ ID NO: 2) to efficiently convert 4-hydroxybenzyl alcohol into gastrodin using an E. coli host (FIG. 1). Furthermore, bypassing early biosynthetic steps that have previously been employed favors of a single-step biotransformation process in which 4-hydroxybenzyl alcohol is directly fed to a microorganism that is expressing a single UGT gene.

This enzyme has not been previously described in literature. The present disclosure describes the cloning and use of GeUGT to produce gastrodin at high titers using a biotransformation approach by feeding 4-hydroxybenzyl alcohol. Furthermore, 11 UGT enzymes that have not previously been described as enzymes that convert 4-hydroxybenzyl alcohol to gastrodin are identified and described herein. SiUGT (SEQ ID NO: 4); CaUGT (SEQ ID NO: 6); PpUGT (SEQ ID NO: 8); NbUGT1 (SEQ ID NO: 10); NbUGT2 (SEQ ID NO: 12); PcUGT (SEQ ID NO: 14); WsUGT (SEQ ID NO: 16); AtUGT1 (SEQ ID NO: 18); AtUGT2 (SEQ ID NO: 20); PtUGT (SEQ ID NO: 22); and RrUGT (SEQ ID NO:24).

From this search, 5 candidate sequences were selected, transformed into E. coli (′, and tested for enzyme activity. Of these sequences, one was discovered to have significant 4-hydroxybenzyl alcohol UGT activity and named GeUGT (SEQ ID NO: 2). To further explore additional enzymes that could produce gastrodin, a BLAST search of these sequence was performed against publicly available sequences in the NCBI database. Fifteen additional enzymes were selected to test. Of these enzymes, 11 were capable of producing gastrodin at varying efficiencies (SEQ ID NOs: 4, 6, 8, 10, 12, 14, 16, 18, 20, 22, and 24; FIG. 4). None of the 11 enzymes have been reported previously as UGTs that are capable of transforming 4-hydroxybenzyl alcohol into gastrodin.

After assessment of gastrodin production efficiency, it was clear that the GeUGT was the most efficient at converting 4-hydroxybenzyl alcohol to gastrodin, with no 4-hydroxybenzyl alcohol remaining after 48 hours. While the enzyme from R. rosea also converted all 4-hydroxybenzyl alcohol into gastrodin, GeUGT showed a greater conversion rate at 24 hours after inoculation.

### Example 2

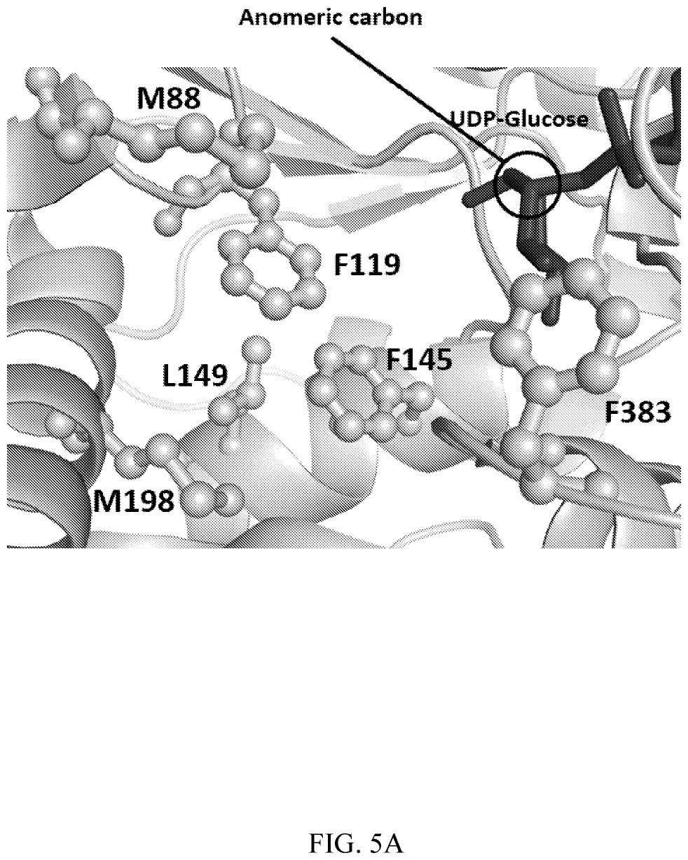

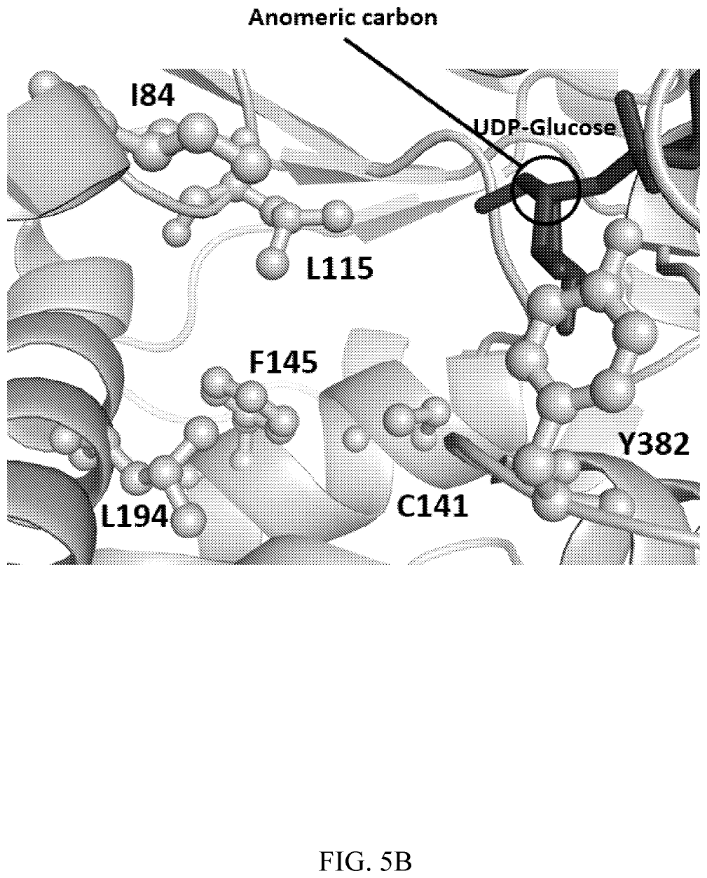

To understand the rate of GeUGT, the biochemical basis for this activity was examined. Due to the lack of an experimental crystallographic structure of AsUGT, the publicly available predicted structure of AsUGT from the Alphafold protein structure database was used to generate PyMOL 3D visualizations of the GeUGT active site (FIG. 5A) and the AsUGT active site (FIG. 5B). This allowed a closer look at the active site surrounding the region in which UDP-glucose is bound. By superimposing this model with a closely related experimental structure that contained UDP-glucose (2VCE), it was possible to approximate UDP-glucose's location in AsUGT, since the binding of UDP-glucose is well conserved within the GT1 superfamily of glycosyltransferases. Finally, all active site residues in AsUGT were converted to the corresponding GeUGT residues based on the sequence alignment by modifying the model in Pymol.

A clear difference in the structure of the active site was observed, providing an answer to why GeUGT is much more efficient than AsUGT at producing gastrodin. In order for gastrodin to be biosynthesized, the phenolic oxygen of hydroxybenzyl alcohol must be in close proximity to the anomeric carbon of UDP-glucose. In the case of 4-hydroxybenzyl alcohol, this means that the hydroxybenzyl moiety should also be in close proximity to UDP-glucose. In the GeUGT model, a concentration of aromatic side-chains in close proximity to the UDP-glucose molecule is observed, in particular at F119, F145, and F383. Strikingly, in AsUGT, L115 and C141 (corresponding to F119 and F145 in GeUGT) are the residues having close proximity to the UDP-Glucose molecule, which is not ideal for ensuring the hydroxybenzyl moiety is bound in the correct position. AsUGT is known for being a broad-substrate UGT, while the GeUGT is likely tailored specifically to glycosylate 4-hydroxybenzyl alcohol, potentially through these aromatic residues that cluster close to the binding site of UDP-glucose. Although phenylalanine (F) was identified at 119, 145, and 383; other amino acids having aromatic hydrophobic side chains (e.g., tyrosine (Y) and/or tryptophan (W)) are contemplated by the present disclosure. Similarly, the present disclosure provides for the use of an amino acid having a hydrophobic side chain with another amino acid having similar chemical properties (e.g., leucine (L) and/or methionine (M)).

Variability in the active site was obtained from the alignment of all the UGTs identified in Example 1. At each position, the difference between AsUGT and GeUGT was observed. Next, all of the other sequences were examined to see whether the corresponding residue was more closely related to AsUGT or GeUGT. In some cases, other UGTs have residues that are similar to GeUGT and different than AsUGT, and thus some of the residues have alternatives added to them.

### INCORPORATION BY REFERENCE

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A host cell comprising a transgene encoding a heterologous uridine 5′-diphospho-glucosyltransferase (UGT) operably linked to a promoter, wherein the heterologous UGT comprises an amino acid sequence comprising SEQ ID NO: 2.

2-30. (canceled)

31. The host cell of claim 1, wherein the host cell is a plant cell, a fungal cell, a yeast cell, an insect cell, or a bacterial cell.

32. The host cell of claim 1, wherein the heterologous UGT is codon-optimized for expression in the host cell.

33-58. (canceled)

59. A method of producing gastrodin in a host cell, the method comprising culturing the host cell in cell culture medium comprising 4-hydroxybenzyl alcohol, wherein the host cell expresses a transgene that encodes a heterologous uridine 5′-diphospho-glucosyltransferase (UGT) operably linked to a promoter, wherein the heterologous UGT comprises an amino acid sequence comprising SEQ ID NO: 2.

60-88. (canceled)

89. The method of claim 59, wherein the host cell is a plant cell, a fungal cell, a yeast cell, an insect cell, or a bacterial cell.

90. The method of claim 59, wherein the heterologous UGT is codon-optimized for expression in the host cell.

91-116. (canceled)

117. The method of claim 59, wherein the cell culture medium further comprises glucose.

118. The method of claim 59, further comprising making the host cell, the method comprising introducing a vector into the host cell, the vector comprising a nucleic acid encoding the heterologous uridine 5′-diphospho-glucosyltransferase (UGT) operably linked to the promoter.

119. The method of claim 59, wherein the gastrodin is extracted using maceration, percolation, decoction, reflux extraction, soxhlet extraction, pressurized liquid extraction, supercritical fluid extraction, ultrasound assisted extraction, pulsed electric field extraction, enzyme assisted extraction, hydro distillation, steam distillation, or any combination thereof.

120. The method of claim 59, wherein the concentration of gastrodin within the cell culture medium after 24 h incubation is at least about 4 mM, 6 mM, or 8 mM.

121. The method of claim 59, wherein the concentration of 4-hydroxybenzyl alcohol within the cell culture medium after 24 hr incubation is not greater than 2 mM, 1.5 mM, or 1 mM.

122. A vector comprising a nucleic acid encoding a uridine 5′-diphospho-glucosyltransferase (UGT) for converting 4-hydroxybenzyl alcohol into gastrodin, wherein the uridine 5′-diphospho-glucosyltransferase (UGT) comprises SEQ ID NO: 2.

123. The vector of claim 122, wherein the nucleic acid encoding the uridine 5′-diphospho-glucosyltransferase (UGT) for converting 4-hydroxybenzyl alcohol into gastrodin has at least about 95% nucleotide sequence identity to SEQ ID NO: 1.

124-186. (canceled)

187. A method of making a transgenic host cell, the method comprising introducing a vector into a host cell, the vector comprising a nucleic acid encoding a heterologous uridine 5′-diphospho-glucosyltransferase (UGT) operably linked to a promoter, wherein the heterologous UGT comprises an amino acid sequence comprising SEQ ID NO: 2.

188-215. (canceled)

216. The method of claim 187, wherein the host cell is a plant cell, a fungal cell, a yeast cell, an insect cell, or a bacterial cell.

217. The method of claim 187, wherein the heterologous UGT is codon-optimized for expression in the host cell.

218-244. (canceled)


SEQUENCE LISTING (28 sequences)

SEQ ID NO: 1 - DNA, 1449 bp, Gastrodia elata
  source 1..1449: mol_type = genomic DNA
atggagattccatccgaaggtagacgcccgcatcttgccatggtcagtagccccggaatgggccacctgttccctcttgctgagttagctaagattctggttaacagacatagattcaccgtaacatttattacctttgcggcgagcgacaaccgtgcagctcacactttcttatcgactttgccctcagggatttcttggctcaccctgccaccagtgcctttagacgacattccgccagccgctacaattgagactcgcatgtctttagtttcgcttcggagtctgccggccatccgaagcgccttagccgacttgcactcaacgacgaacttggttgctgtggttgctgacttctttgcgactgatgcttttgcagcggcacgtgacatcggtgttccctactatatgtttatcccaacaaacttattgttcttgtctcttctgttagagctgccagcattagatgctgccattacaggtcggttccaggacttagaaaccccgttacgtctgtctggctgcgttccgattcccggctcagacattttgacaccccttcttaatagatccagcgaggagtacagatggatgttacaccatgcgcgcctgtacagacaagcgcaagggattttggttaattcttttgcagctattgaaccaggaccgacaaagagccttagccaaaattccttatcacgtccaccggtatacccggtggggccaatggttcaatcgcccaagccgggaacgagcttatgccttgcgtggctggatcgccagcccgagggttcggttttattcgtttccttcggctctggtggcacattaactagaacccaactcggggagctggccctgggtctggaagcatccaagcaacgcttcctgcttgtagtaagatcaccgtgcgacgaagatagctcgtccacgtatttcacagcacaaacaaaggacgacccgttgttattcttgcccgaagggtttgtggaccgtacccgggacgtgggtctggtggtaccgtcgtgggctccgcaaattgaagtcctggcgcatcgtgcgacaggcgggttcttaagtcattgcggatggaacagcacattggaatcggtcagtcacggcgttcctcttatagcctggccgctgttcgcagaacaattcatgaatgcagtcatgctggtggaagaagcccgcgtcgccatgcggcccaagaaggcagatgctgcggcagctgtagatagagctgaggtatcacgagtcgcagcagcgctgatggaaggcgaagaaggcaaggcactgcggcgtcgagcccaacagttgaaggaagcggcacacgccgccttaagagagggcggcagtagttacgaagccctcggcttagtggctgacatgtggaaggagaagagtgctggggcaggtggagccgccgccgcagtggcaccccaataa

SEQ ID NO: 2 - protein, 482 aa, Gastrodia elata
MEIPSEGRRPHLAMVSSPGMGHLFPLAELAKILVNRHRFTVTFITFAASDNRAAHTFLSTLPSGISWLTLPPVPLDDIPPAATIETRMSLVSLRSLPAIRSALADLHSTTNLVAVVADFFATDAFAAARDIGVPYYMFIPTNLLFLSLLLELPALDAAITGRFQDLETPLRLSGCVPIPGSDILTPLLNRSSEEYRWMLHHARLYRQAQGILVNSFAAIEPGPTKSLSQNSLSRPPVYPVGPMVQSPKPGTSLCLAWLDRQPEGSVLFVSFGSGGTLTRTQLGELALGLEASKQRFLLVVRSPCDEDSSSTYFTAQTKDDPLLFLPEGFVDRTRDVGLVVPSWAPQIEVLAHRATGGFLSHCGWNSTLESVSHGVPLIAWPLFAEQFMNAVMLVEEARVAMRPKKADAAAAVDRAEVSRVAAALMEGEEGKALRRRAQQLKEAAHAALREGGSSYEALGLVADMWKEKSAGAGGAAAAVAPQ

SEQ ID NO: 3 - DNA, 1431 bp, Sesamum indicum
  source 1..1431: mol_type = genomic DNA
atggaaaccgcctccgccgccccgtccccatcccacatcgccgtccttccctcccccggcatgggtcaccttatccccatcgttgaattcgccaagaaactcctccatcaccaccgtatctccaccactctcatcatgcccactgatggccccctctccaatgcccaaaataccctcttttccaccctcccccccggcatagattacattctcctccccaccctcaacttcgacgacttgccgcccgacgtcaagatcgagactcgtatttcgctcaccgttactcgttccctgccgtcacttcgtgacgcggttaagtccctgctcgagaggacgccgctcgctgctttggtggtcgatcttttcgggaccgatgggtttgaagtcgctcttgaattcaaaatccctccttatattttcttcccttccaccgctatggctctgtctctgtttctatacctgcccaagctggacgaaacggtgtcgtgtgagtatagggaggtggctgagaagttgctgattccgggttgtactccgatcgacggccgggatttgctggatccgcttcaggataggaagaatgatgcgtataaatgggttcttcatcatacaaagaggtacaggatggctgaagggataatcttgaatagctttaaagaactggagcctggtgcgattgaagctttgcaagaaaaggaagacagaaagccagttgtttatcccattgggcctttgatccaaacgggcccaaaacccgaggtcgatgaatcgtgcgtttgtctgaaatggctggatgagcagcccagaggctcggtcttgtatgtctctttcgggagtggtgggactttgtctcatgatgagatggttgagctcgcattggggttagaaatgagtgagcagagatttttgtgggtcgctagatgtccaaacgacacaacagccaatgcaacatattttaatattcagagttctaatgaccctctggcttatttgccagatgggttcttggagaggaccaagggaagggggctggtggtgcccacatgggccccacaggctcatatactcggccatgaatcaacgggtgcatttctaacacattgtggatggaactcgattctagagagcgtagtgaatggagtgccgatgatagcctggccgttgtatgctgaacaacggatgaacgccctgatgcttcatgaggacgtgcgagtggccttgaggcctaaagccggagaaaatggctttgtgggacgagatgaaatagcgaaaacagtgaaaggtttggtggaaggggaagaagggaaggagattcggagtcggatgagggaccttaaggatgctgctgcaaaagtgcttagcgtaaatggttcgtccacaaagatactagctgaattggcaggcagatggaagagtaacacttgcatataa

SEQ ID NO: 4 - protein, 476 aa, Sesamum indicum
METASAAPSPSHIAVLPSPGMGHLIPIVEFAKKLLHHHRISTTLIMPTDGPLSNAQNTLFSTLPPGIDYILLPTLNFDDLPPDVKIETRISLTVTRSLPSLRDAVKSLLERTPLAALVVDLFGTDGFEVALEFKIPPYIFFPSTAMALSLFLYLPKLDETVSCEYREVAEKLLIPGCTPIDGRDLLDPLQDRKNDAYKWVLHHTKRYRMAEGIILNSFKELEPGAIEALQEKEDRKPVVYPIGPLIQTGPKPEVDESCVCLKWLDEQPRGSVLYVSFGSGGTLSHDEMVELALGLEMSEQRFLWVARCPNDTTANATYFNIQSSNDPLAYLPDGFLERTKGRGLVVPTWAPQAHILGHESTGAFLTHCGWNSILESVVNGVPMIAWPLYAEQRMNALMLHEDVRVALRPKAGENGFVGRDEIAKTVKGLVEGEEGKEIRSRMRDLKDAAAKVLSVNGSSTKILAELAGRWKSNTCI

SEQ ID NO: 5 - DNA, 1413 bp, Coffea arabica
  source 1..1413: mol_type = genomic DNA
atggatcaaacaccccgtgtagccctcttaccatctcccgggatgggtcatctcatcccgctggtggagtttgctaagcgccttattctccaacacgacttctcagtcaccataattctcccgacagatggacctctctctaaggcacaaaccacctttcttgctgctcttcctgctgccatcgattacattcttcttcctcctgttaacttcgacgacttagctgacgacgttaggattgagacgcgtatttccctcaccgtcactcgctctcttccttcccttcgtgatgcgctcaagtcgttggttgacaccgccaagttggccgctctcgtggtcgatcttttcggcacggatgcctttgatgtagccaatgaattcaagctcccgccatatatcttcttcccttccaccgccacggctttggcgtttttctcctacctgcctaagcttcatgagatggttgcatgtgaatttagagacttgcccgggccaattcgagtcccgggttgtgtaccaattcacgggagggatctcctcgacccggctcaggaccgaaagaacgacgcctacaagtggctgcttcaccacgcgaggaggtacagcctggccgagggaatcgtggtgaacagcttcaaggacttggagcctggacctttaaaagcgttgcaggagcaagaaccgggtaagccaccggtttatccggttgggccacttatacagatgggctcgggggagaagaggggggaggagccggaatgcttgaaatggttggatgatcagcccagcgggtccgttctgtacatttcgtttgggagcggtgggaccctctcccataatcagctagtcgagctagcattcggattggagatgagcgagcaaagattcttgtgggtggtccggagtccaaatgatggagttgccaatgccacttacttcaccgtcaacagtcaaaatgatcctctggcttttatgccggaagggttcttggataggatcaagggtcgaggttttttggtgccatcttgggctccacaggctaaaattctcggccacagttccaccggcgggtttctgacccactgcggttggaactcgactcttgaaagcgtggttgaggggattccgcttattgcttggccgctctacgcggagcagaagatgaacgcagtaatgcttgcggaggatctaaaagttgcttcgaggccaaaggctgacgaaaatggattcgtgggaagagttgagattgcaaacatggtcaagaggttaatggaaggagaagaagggaaggggctccgtagccgaatgaaggagctcaaggatgcagctgctaaggttttgagccaagacggttcttccacagaagctctagcccaagttgccggcaagtggcaggccaaaatttgtacgtaa

SEQ ID NO: 6 - protein, 470 aa, Coffea arabica
MDQTPRVALLPSPGMGHLIPLVEFAKRLILQHDFSVTIILPTDGPLSKAQTTFLAALPAAIDYILLPPVNFDDLADDVRIETRISLTVTRSLPSLRDALKSLVDTAKLAALVVDLFGTDAFDVANEFKLPPYIFFPSTATALAFFSYLPKLHEMVACEFRDLPGPIRVPGCVPIHGRDLLDPAQDRKNDAYKWLLHHARRYSLAEGIVVNSFKDLEPGPLKALQEQEPGKPPVYPVGPLIQMGSGEKRGEEPECLKWLDDQPSGSVLYISFGSGGTLSHNQLVELAFGLEMSEQRFLWVVRSPNDGVANATYFTVNSQNDPLAFMPEGFLDRIKGRGFLVPSWAPQAKILGHSSTGGFLTHCGWNSTLESVVEGIPLIAWPLYAEQKMNAVMLAEDLKVASRPKADENGFVGRVEIANMVKRLMEGEEGKGLRSRMKELKDAAAKVLSQDGSSTEALAQVAGKWQAKICT

SEQ ID NO: 7 - DNA, 1437 bp, Paris polyphylla
  source 1..1437: mol_type = genomic DNA
atggatagtgataacattaaagccccgttggccgccacacacatcgccgtcttcccgacgccggggatgggccacctcattcctctcgccgagctggcgaaaagcctcgtcgcccgccatggattctcggtcaccttcatcaccctcgccatctccgcctccaaagcccagtctgcgttcctcgacaccctcccaccctcgatcgcgtccctctcccttcctgccgtccccctcgacgatctcccggccgatgctgccatcgagacggtgatctcagtcgccactgatcgatcccttcccgccctccgcgagctcctcctcgacttaaagcggtcggcaaaactcgtcgctttcgtggctgatcttttcggcgccgacaccttcgctgttgcagctgcgattggggtctcgccgtaccttttccatccttctaattttcagacggtctctctgatgctctcgctcccgcggcttgatgctgagacccagggacagttcagggacctccacgagccggtccggctgccgggttgtattcctatcccgggggcagagctcctccatccgctccaggaccgcaccatcgaggtgtacagatggatggtgcaccacggggagaagtaccgcgatgctgaggggatcatcgtcaacacctttgaggaaatcgagccttgggcatcggagttcttctacaagaaggcgggaccagagtggccaccaatttatccagtgggtccagtgatccaggccgaggggtcgggggtcggattgagcgggtcagagtgtttggcgtggctcgacgggcagcccaagggttcggtgatttttgtgtcattcgggagcgctggcacgctgtcgacggagcagacgacagagctggcgacagggctggagatgagcgggcagcgattcttgtgggtggtgcggaagcccaatgatggaggctcgagcgcgagatacttcacctctggggtttcagaagatccatacgagtttctgccgaaggggttcatcgaccggatcaaggggacgggaatgctggtgtcgtcgtgggcgccacaggtgcaggtgctgggccacccgtcgacaggtggattcctgtcccactgcggatggaactcaaccctggagagcgtgtcgcacggggtgccgatgatcacatggcctctttttgcggagcagaggcagaatgcagtgatgctggtggagggggtgggagtggcgctgagtctcgaggccggagaggacggggtttataagagcgaggagatcgcgagggtggtgaagggattgatggagggggaagaggggaaggtgatgcgacataaggcgagggagctggaggcggcagcggcgaggactctggcccaagggggggcgtcctacgaggcgctcaatgaagtcgctaacaagtggaaggcttcggctgttgcttag

SEQ ID NO: 8 - protein, 478 aa, Paris polyphylla
MDSDNIKAPLAATHIAVFPTPGMGHLIPLAELAKSLVARHGFSVTFITLAISASKAQSAFLDTLPPSIASLSLPAVPLDDLPADAAIETVISVATDRSLPALRELLLDLKRSAKLVAFVADLFGADTFAVAAAIGVSPYLFHPSNFQTVSLMLSLPRLDAETQGQFRDLHEPVRLPGCIPIPGAELLHPLQDRTIEVYRWMVHHGEKYRDAEGIIVNTFEEIEPWASEFFYKKAGPEWPPIYPVGPVIQAEGSGVGLSGSECLAWLDGQPKGSVIFVSFGSAGTLSTEQTTELATGLEMSGQRFLWVVRKPNDGGSSARYFTSGVSEDPYEFLPKGFIDRIKGTGMLVSSWAPQVQVLGHPSTGGFLSHCGWNSTLESVSHGVPMITWPLFAEQRQNAVMLVEGVGVALSLEAGEDGVYKSEEIARVVKGLMEGEEGKVMRHKARELEAAAARTLAQGGASYEALNEVANKWKASAVA

SEQ ID NO: 9 - DNA, 1437 bp, Nicotiana benthamiana
  source 1..1437: mol_type = genomic DNA
atggcggaaactccaataacagcacaatcagaacaaaaaccccatgtcgtccttttaccaactccaggaatgggtcacttaatccccctcgttgaattcgccaagcgactcattcacgagcacgacctctctataacaatcatccttcctactaacggtcccatctctaaatctcaaaacaaattcctcaactctcttccttctaccatgaattacctccttttacctcccgttaacttcgatgatttacctgacgacgttaaaatcgagacacgtatctctcttacagtcacacgttcactttcttcttttcgtaaagttttcatgttactagttgaaacgaagaaaatagttgctttagtggtcgatctttttggcacggacacatttgatatagcaaaagaatttaatgtctctccttatattttttaccctactacagctatgtgcttatcattatttctttatttgcctacacttgatgtgagtgtgtcatgtgagtatagagagttgaaaggaccggttcggattccgggttgtgttcctatagaagcaaaggaacttctggacccgattcaggataggaagaatgatgcttacaaatggctgcttcatcatactaagaggtataaaatggcggagggaatagtggtgaatagttttaaggagttggaaccaggtgccattaatgatttgcatgaggaaaaaccggataagccgccagtttacgcagttggaccgcttatacatatggatgtaagcagtaaagctgaggaattagaatgtttaaaatggctagatgagcagccatgtaactcagtcttgtacatttcctttggaagtggtggaactctctcacatgagcagctaattgaacttgcaatgggtttagaaatgagtgagcaaagatttctatgggttattagatgcccaaatgatagaattgctaatgccacttactttaatattcaaagttcaagtaacccttttgattctttacctaaagggttcttggaaagaaccaaaggacaaggattaatggtacctaattgggccccacaagcccaaatccttagccatgggtcgacaggcgggttcttgactcactgtggatggaactcgacgttagagagtgtggtccatggcattccacttattgcttggccattatatgcggaacaaagaatgaacgcgataatgctaaacgaggaattaaaagtggcactgaggctggaaacaagtgagaatggcatcgtgggaagagaggaaattgctcaagtggtaagagaattgatggaaggtgaagaaggaaaaggagttcgtagtagaatgagagatttaaaagatgaagctgctaaagtacttggtgaaaatggttcctctacaaaagcactagctgaattggcttccaaaatgagaaatgtgttacaaaattaa

SEQ ID NO: 10 - protein, 478 aa, Nicotiana benthamiana
MAETPITAQSEQKPHVVLLPTPGMGHLIPLVEFAKRLIHEHDLSITIILPTNGPISKSQNKFLNSLPSTMNYLLLPPVNFDDLPDDVKIETRISLTVTRSLSSFRKVFMLLVETKKIVALVVDLFGTDTFDIAKEFNVSPYIFYPTTAMCLSLFLYLPTLDVSVSCEYRELKGPVRIPGCVPIEAKELLDPIQDRKNDAYKWLLHHTKRYKMAEGIVVNSFKELEPGAINDLHEEKPDKPPVYAVGPLIHMDVSSKAEELECLKWLDEQPCNSVLYISFGSGGTLSHEQLIELAMGLEMSEQRFLWVIRCPNDRIANATYFNIQSSSNPFDSLPKGFLERTKGQGLMVPNWAPQAQILSHGSTGGFLTHCGWNSTLESVVHGIPLIAWPLYAEQRMNAIMLNEELKVALRLETSENGIVGREEIAQVVRELMEGEEGKGVRSRMRDLKDEAAKVLGENGSSTKALAELASKMRNVLQN

SEQ ID NO: 11 - DNA, 1437 bp, Nicotiana benthamiana
  source 1..1437: mol_type = genomic DNA
atggcggaaactgctatagtaacaaaatcagaaaatccccatatcgtaattttaccaagtcctggtatgggtcacttaatccctcttgttgaattttctaagagactaatctctcaacaccagttttctgtcactcttattctccctacagatggacctatctctaattctcagaaaattttccttaattctcttccctcttgcatggattatcaccttcttcctcctgttaatttcgatgatttgcctcttgatgttaaaattgaaactagaatctcccttactgttacgcgttctctttcttcacttcgtgatgtttttaaaacacttgttgaatccaagaaagtagttgcttttgtggttgatctttttggtactgatgcatttgatgttgccattgattttaacgtttccccttatatttttttcccttctactgccatggctttgtctctgtttctttacttgcctaagcttgatgcaaccgtgtcatgtgagtacagagacttacctgacccgattcagattccgggttgtattcctatttatggcaaggatcttctcgacccggttcaggaccggaaaaacgaagcttacaaatggcttcttcatcattctaagaggtatggaatggctgagggagtaatagctaacagtttcaaggaattggaaggaggggcaattaaagctctgcaggaagaagaacccggcaagccacccgtttacccggttggaccactgattcagatggattcgggtagtaatgttgacgggtcgggttgtctgacgtggctggatgaacagccacttggctctgttttgtatatctcgtacgggagtggtgggaccctctctcatgagcagctcattgaagttgcatcaggactggaaatgagtgaacaaaggttcttgtgggtaattagatgtccagatgacactgtagccaatgccacttattttaatgtccaagattcaacaaaccctcttgattttctcccaaaagggttcttggaaaggaccaaaagtttaggtttagtggtgcctaattgggccccacaagctcaaattcttgcccatggttcaacgggcgggtttttgactcattgcggatggaactcgattctggaaagtgtgatccacggagtaccactcatagcttggccactttatgctgaacaaaagatgaatgctgttatgctagctgaggatattaaagtagcactgaggccaaaagccaatgaaaatggactaatgggaagattggaaattgctaaagtagtgaaaggattaatggaaggtgaagaaggaaaaggagtgcgcactagaatgagagaccttaaagatgcagcagctaaagttctaagtcaagatggttcttctacaaaagcattagctgaattggctactaaattgaagaataaggtgttatacaattaa

SEQ ID NO: 12 - protein, 478 aa, Nicotiana benthamiana
MAETAIVTKSENPHIVILPSPGMGHLIPLVEFSKRLISQHQFSVTLILPTDGPISNSQKIFLNSLPSCMDYHLLPPVNFDDLPLDVKIETRISLTVTRSLSSLRDVFKTLVESKKVVAFVVDLFGTDAFDVAIDFNVSPYIFFPSTAMALSLFLYLPKLDATVSCEYRDLPDPIQIPGCIPIYGKDLLDPVQDRKNEAYKWLLHHSKRYGMAEGVIANSFKELEGGAIKALQEEEPGKPPVYPVGPLIQMDSGSNVDGSGCLTWLDEQPLGSVLYISYGSGGTLSHEQLIEVASGLEMSEQRFLWVIRCPDDTVANATYFNVQDSTNPLDFLPKGFLERTKSLGLVVPNWAPQAQILAHGSTGGFLTHCGWNSILESVIHGVPLIAWPLYAEQKMNAVMLAEDIKVALRPKANENGLMGRLEIAKVVKGLMEGEEGKGVRTRMRDLKDAAAKVLSQDGSSTKALAELATKLKNKVLYN

SEQ ID NO: 13 - DNA, 1416 bp, Phacelia campanularia
  source 1..1416: mol_type = genomic DNA
atggaggaaacaacccacatagctctattaccaacaccagggatgggtcatctcatcccactcgttgaattcgcgaaacgtctaattcaactcgaacactacaaattctcagttactctactcctccccacaactggaactgtttcagactctcaactgctgttccttgaagctttacccaatgggatatcatacatagctcttccacctataaacgtcgatgatttatcaagtgttgttgctgaaacccgcatagctcttactataactcgctctcttcctgctgtggaagatgctttgaagtctttggttagcgagaaaaaggtggttgctctggtggttgatcttttcgggactgatggttttgaagtcgcgaaggcgttcggtataagtccatacatttttttccctacagcagctattaatttatctctatttcttcacatgtctgagcttgataagaggattacatgtgagtatagagatatggatgagcctgttaagcttccagggtgtataccaatcaatggtaaagatttgctcgaccctcttcaagataggacaaatgatgcatataaatggactcttcaccatgcgagtaggtataaattagctgaagggatagttgtgaatacttttgaggatttagagccagaggctataaaagctttacatgaagaacatggtgtgccaccattgtttcctgtaggaccattaacacagaaggttttgagctcgaatggcgctgaagaatcggattgttgtttgaaatggttgaatgatcaaccactcgggtccgttttatttgtgtcttttggtagtggtggaacgcttacggaagaacaatttctcgaattagccacagggttggaattaagtgaacaaagattcatatgggttgttagaagtccaaataaagttgctaatgctgcttatttcaatcaattagatcaagatccatttccatttttaccagaagggtatttagatagggttaaagggcgtggtttagttgtagcagattgggcaccgcaagcaaagattcttaaccatggttcaacaggtggatttttaacgcattgtgggtggaattcgattcttgaaagtgtggttagtgggataccattagttgcttggccactttatgcagaacagagaacaaatgctgtgatgttatctgaaggcttgaaggttgctttaaggcctaaaagtgttggtgaaaatggattgatcgatagagttgaaatcgctcgtgttgttaaaggcttgtttgtcggtgaagagggtacgcagatttgcagcagaatgagagatttgaaagatgcttctgtgaaagttcttggcgaagatggttcttcaagaaaatcacttcttgaattggcttcaaaatggaagagcaagactgtttctgaataa

SEQ ID NO: 14 - protein, 471 aa, Phacelia campanularia
MEETTHIALLPTPGMGHLIPLVEFAKRLIQLEHYKFSVTLLLPTTGTVSDSQLLFLEALPNGISYIALPPINVDDLSSVVAETRIALTITRSLPAVEDALKSLVSEKKVVALVVDLFGTDGFEVAKAFGISPYIFFPTAAINLSLFLHMSELDKRITCEYRDMDEPVKLPGCIPINGKDLLDPLQDRTNDAYKWTLHHASRYKLAEGIVVNTFEDLEPEAIKALHEEHGVPPLFPVGPLTQKVLSSNGAEESDCCLKWLNDQPLGSVLFVSFGSGGTLTEEQFLELATGLELSEQRFIWVVRSPNKVANAAYFNQLDQDPFPFLPEGYLDRVKGRGLVVADWAPQAKILNHGSTGGFLTHCGWNSILESVVSGIPLVAWPLYAEQRTNAVMLSEGLKVALRPKSVGENGLIDRVEIARVVKGLFVGEEGTQICSRMRDLKDASVKVLGEDGSSRKSLLELASKWKSKTVSE

SEQ ID NO: 15 - DNA, 1440 bp, Withania somnifera
  source 1..1440: mol_type = genomic DNA
atggcggaaacccctatgactgctacagaaacacaaaatacccatattgcaattttaccttctcctggtatgggtcacttaatccctcttgttgaattttctaaaagactcatccttcgacaccaattttctgttactcttattctccctactgatggtcctatctctaattctcagaaacttttccttaattcactcccttcttccatggattatcaccttcttcctcctgtgaattttgatgatttacctctagatgttaaaatcgaaactcgtatttcccttactgttacgcgttctttatcttcactacgtgaagtttttaagacacttgttgaggaaaagaaaacagttgctcttgtggttgatcttttcggcactgatgcatttgatgttgctattgactttaaggtttccccttatatattttttccttctactgcaatggctttgtgtttgttttgttacttgcataaacttgatgaaatggtgtcgtgtgagtatagagacatgtctgaacctgttcagattacgggttgcatccctatccatggaaaggatcttcttgacccagttcaggaccggaaaaatgaagcttacaaatgggttcttcatcattcgaagaggtataggatggcagagggaatagtagcaaacagctttaaggaattggaaggaggagctattagagctttgcaagaagaagaaccgggtaaaccgccggtttacccggttggaccgatcattcagatggattcgggtaataaggttgatgagtcggattgtctgacgtggctggatgaacagccacgtggctctgtgttatacatttcgtatgggagtggtgggactctctctcatcagcaggtcattgaactcgcatcaggcttggagatgagtgagcaaaggttcttgtgggtaatcagatgccctaatgacagaatagccaatgctacttactttagtgtccaagattcaactaaccctcttgatttcctaccaaaagggttcttggaaaggaccaaagggttaggtttagtggtgcccaattgggccccacaagcccaaatcttggcccacgactcaactggcgggttcttgactcactgcggatggaactcagctctagaaagcgtggtccacggggtaccgctgatagcttggccactatatgcagagcaaagaatgaatgcggttatgctatctgaggacataaaagtagcactaaggccaaaagtcaatgaagaaaatggactagtgggaagattggaaattgctaaagttgtgaaaggactgatggaaggtgaagaaggaaaaggagtgcgcagtagaatgagagaccttaaagatgcagcagccaaagtgcttagtgatgatggttcttctacaaaagcactagctgaattggctactaaaatgaagaaaagaagtgtccaataa

SEQ ID NO: 16 - protein, 479 aa, Withania somnifera
MAETPMTATETQNTHIAILPSPGMGHLIPLVEFSKRLILRHQFSVTLILPTDGPISNSQKLFLNSLPSSMDYHLLPPVNFDDLPLDVKIETRISLTVTRSLSSLREVFKTLVEEKKTVALVVDLFGTDAFDVAIDFKVSPYIFFPSTAMALCLFCYLHKLDEMVSCEYRDMSEPVQITGCIPIHGKDLLDPVQDRKNEAYKWVLHHSKRYRMAEGIVANSFKELEGGAIRALQEEEPGKPPVYPVGPIIQMDSGNKVDESDCLTWLDEQPRGSVLYISYGSGGTLSHQQVIELASGLEMSEQRFLWVIRCPNDRIANATYFSVQDSTNPLDFLPKGFLERTKGLGLVVPNWAPQAQILAHDSTGGFLTHCGWNSALESVVHGVPLIAWPLYAEQRMNAVMLSEDIKVALRPKVNEENGLVGRLEIAKVVKGLMEGEEGKGVRSRMRDLKDAAAKVLSDDGSSTKALAELATKMKKRSVQ

SEQ ID NO: 17 - DNA, 1443 bp, Arabidopsis thaliana
  source 1..1443: mol_type = genomic DNA
atggaggaatccaaaacacctcacgttgcgatcataccaagtccgggaatgggtcatctcataccactcgtcgagtttgctaaacgactcgtccatcttcacggcctcaccgttaccttcgtcatcgccggcgaaggtccaccatcaaaagctcagagaaccgtcctcgactctctcccttcttcaatctcctccgtctttctccctcctgttgatctcaccgatctctcttcgtccactcgcatcgaatctcggatctccctcaccgtgactcgttcaaacccggagctccggaaagtcttcgactcgttcgtggagggaggtcgtttgccaacggcgctcgtcgtcgatctcttcggtacggacgctttcgacgtggccgtagaatttcacgtgccaccgtatattttctacccaacaacggccaacgtcttgtcgttttttctccatttgcctaaactagacgaaacggtgtcgtgtgagttcagggaattaaccgaaccgcttatgcttcctggatgtgtaccggttgccgggaaagatttccttgacccggcccaagaccggaaagacgatgcatacaaatggcttctccataacaccaagaggtacaaagaagccgaaggtattcttgtgaataccttctttgagctagagccaaatgctataaaggccttgcaagaaccgggtcttgataaaccaccggtttatccggttggaccgttggttaacattggtaagcaagaggctaagcaaaccgaagagtctgaatgtttaaagtggttggataaccagccgctcggttcggttttatatgtgtcctttggtagtggcggtaccctcacatgtgagcagctcaatgagcttgctcttggtcttgcagatagtgagcaacggtttctttgggtcatacgaagtcctagtgggatcgctaattcgtcgtattttgattcacatagccaaacagatccattgacatttttaccaccgggatttttagagcggactaaaaaaagaggttttgtgatccctttttgggctccacaagcccaagtcttggcgcatccatccacgggaggatttttaactcattgtggatggaattcgactctagagagtgtagtaagcggtattccacttatagcatggccattatacgcagaacagaagatgaatgcggttttgttgagtgaagatattcgtgcggcacttaggccgcgtgccggggacgatgggttagttagaagagaagaggtggctagagtggtaaaaggattgatggaaggtgaagaaggcaaaggagtgaggaacaagatgaaggagttgaaggaagcagcttgtagggtgttgaaggatgatgggacttcgacaaaagcacttagtcttgtggccttaaagtggaaagcccacaaaaaagagttagagcaaaatggcaaccactaa

SEQ ID NO: 18 - protein, 480 aa, Arabidopsis thaliana
MEESKTPHVAIIPSPGMGHLIPLVEFAKRLVHLHGLTVTFVIAGEGPPSKAQRTVLDSLPSSISSVFLPPVDLTDLSSSTRIESRISLTVTRSNPELRKVFDSFVEGGRLPTALVVDLFGTDAFDVAVEFHVPPYIFYPTTANVLSFFLHLPKLDETVSCEFRELTEPLMLPGCVPVAGKDFLDPAQDRKDDAYKWLLHNTKRYKEAEGILVNTFFELEPNAIKALQEPGLDKPPVYPVGPLVNIGKQEAKQTEESECLKWLDNQPLGSVLYVSFGSGGTLTCEQLNELALGLADSEQRFLWVIRSPSGIANSSYFDSHSQTDPLTFLPPGFLERTKKRGFVIPFWAPQAQVLAHPSTGGFLTHCGWNSTLESVVSGIPLIAWPLYAEQKMNAVLLSEDIRAALRPRAGDDGLVRREEVARVVKGLMEGEEGKGVRNKMKELKEAACRVLKDDGTSTKALSLVALKWKAHKKELEQNGNH

SEQ ID NO: 19 - DNA, 1443 bp, Arabidopsis thaliana
  source 1..1443: mol_type = genomic DNA
atggcggaagcaaacactccacacatagcaatcatgccgagtcccggtatgggtcaccttatcccattcgtcgagttagcaaagcgactcgttcagcacgactgtttcaccgtcacaatgatcatctccggtgaaacttcgccgtctaaggcacaaagatccgttctcaactctctcccttcctccatagcctccgtatttctccctcccgccgatctttccgatgttccctccacagcgcgaatcgaaactcgggccatgctcaccatgactcgttccaatccggcgctccgggagctttttggctctttatcaacgaagaaaagtctcccggcggttctcgtcgtcgatatgtttggtgcggatgcgttcgacgtggccgttgacttccacgtgtcaccatacattttctatgcatccaatgcaaacgtcttgtcgttttttcttcacttgccgaaactagacaaaacggtgtcgtgtgagtttaggtacttaaccgaaccgcttaagattcccggctgtgtcccgataaccggtaaggactttcttgatacggttcaagaccgaaacgacgacgcatacaaattgcttctccataacaccaagaggtacaaagaagctaaagggattctagtgaattccttcgttgatttagagtcgaatgcaataaaggccttacaagaaccggctcctgataaaccaacggtatacccgattgggccgctggttaacacaagttcatctaatgttaacttggaagacaagttcggatgtttaagttggctagacaaccaaccattcggctcggttctatacatatcatttggaagcggcggaacacttacatgtgagcagtttaatgagcttgctattggtcttgcggagagcggaaaacggtttatttgggtcatacgaagtccaagcgagatagttagttcgtcgtatttcaatccacacagcgagacagaccccttttcgtttttaccaattgggttcttagaccgaaccaaagagaaaggtttggtggttccatcatgggctccacaggttcaaatcctggctcatccatccacatgcgggtttttaacacactgtggatggaattcgaccttagaaagcattgtaaacggtgtaccactcatagcgtggcctttattcgcggagcaaaagatgaatacattgctactcgtggaggatgttggagcggctctaagaatccatgcgggtgaagatgggattgtacggagggaagaagtggtgagagtggtgaaggcactgatggaaggtgaagagggaaaagccataggaaataaagtgaaggagttgaaagaaggagttgttagagtcttgggtgacgatggattgtccagcaagtcatttggtgaagttttgttaaagtggaaaacgcaccagcgagatatcaaccaagagacgtcccactaa

SEQ ID NO: 20 - protein, 480 aa, Arabidopsis thaliana
MAEANTPHIAIMPSPGMGHLIPFVELAKRLVQHDCFTVTMIISGETSPSKAQRSVLNSLPSSIASVFLPPADLSDVPSTARIETRAMLTMTRSNPALRELFGSLSTKKSLPAVLVVDMFGADAFDVAVDFHVSPYIFYASNANVLSFFLHLPKLDKTVSCEFRYLTEPLKIPGCVPITGKDFLDTVQDRNDDAYKLLLHNTKRYKEAKGILVNSFVDLESNAIKALQEPAPDKPTVYPIGPLVNTSSSNVNLEDKFGCLSWLDNQPFGSVLYISFGSGGTLTCEQFNELAIGLAESGKRFIWVIRSPSEIVSSSYFNPHSETDPFSFLPIGFLDRTKEKGLVVPSWAPQVQILAHPSTCGFLTHCGWNSTLESIVNGVPLIAWPLFAEQKMNTLLLVEDVGAALRIHAGEDGIVRREEVVRVVKALMEGEEGKAIGNKVKELKEGVVRVLGDDGLSSKSFGEVLLKWKTHQRDINQETSH

SEQ ID NO: 21 - DNA, 1434 bp, Persicaria tinctoria
  source 1..1434: mol_type = genomic DNA
atggaatcgccagcagctcctccaactactgctccaccacctcacgtcataatcatgccttcggcaggcatgggacatttgattcctttagccgaatttgccaaaaggttacttcctagattcactttcaccttcgctgtccctaccagtggtccaccatcttcctcgcaaagagatttcctgtcatccttgcccgcttcgattgatacatccttcttgcctgaagttgatctttccgatgctccatcggacgcacaaattgagacactaatgtctttaatggtcgttaggtcactaccttccttgagggatctgatcgcatcttattcagccagcggtaggagggttgcggcattagttgtggatttattcgccacagatgctatcgatgtcgcattggagctgggcatcaggccctttattttctttccatccaccgcaatgacgctttctttctttttacatttggaaaaactagatgaaacagtatcatgtgagttcgcggaattgtcagacccagtccaaataccaggatgcattccagtacatggtaaagatcttattgacccagtacaagatagaaaaaatgatgcttacaagtggttattgcaccattcaaagagatacaagctagcagaaggagtaatcgtaaatagttttgaaggattggagggaggaccaatacgtgaacttttacacccagagccaggcaaacccagggtttacccagttggtcctttgattcaagctggttcttgtgagaaaggtgcggctgcaagaccagaatgtttgaaatggttagatcaacaacctagaggctcagttcttttcgtcaactttggaagtggtggggttttgtcaactgaacagcagaatgaactagccggtgttttggctcatagccaacaaaggttcctttgggtggtcaggccccctaatgacgggatagctaatgcgacatatttttctgtcgacggcgaaatcgatcctttgaaattgctacctgaagggtttctggaacaaacagcaggaaggggtttagtcttgcccatgtgggctccacaaattgatgttttatcgcatgaatctacgggtggattcctaactcactgcggttggaacagcaccttggaatcagtcttccatggtgttccacttatcacgtggcccttatatgctgagcaaaagatgaatgccgttatgttgacagagggattgagagttggcctaaggcctagtgttggtaaggatggcatcattagaggtgatgagattgctagagtaatcggcgaattgatggagggggaagaggggaaaaggataagatcaaaaatgcaggaattaaagcgtgcagcttctgctgttctttctaaagacggttctagcactagagcattggaggaagtggccaagatctgggaatcaaaagtctag

SEQ ID NO: 22 - protein, 477 aa, Persicaria tinctoria
MESPAAPPTTAPPPHVIIMPSAGMGHLIPLAEFAKRLLPRFTFTFAVPTSGPPSSSQRDFLSSLPASIDTSFLPEVDLSDAPSDAQIETLMSLMVVRSLPSLRDLIASYSASGRRVAALVVDLFATDAIDVALELGIRPFIFFPSTAMTLSFFLHLEKLDETVSCEFAELSDPVQIPGCIPVHGKDLIDPVQDRKNDAYKWLLHHSKRYKLAEGVIVNSFEGLEGGPIRELLHPEPGKPRVYPVGPLIQAGSCEKGAAARPECLKWLDQQPRGSVLFVNFGSGGVLSTEQQNELAGVLAHSQQRFLWVVRPPNDGIANATYFSVDGEIDPLKLLPEGFLEQTAGRGLVLPMWAPQIDVLSHESTGGFLTHCGWNSTLESVFHGVPLITWPLYAEQKMNAVMLTEGLRVGLRPSVGKDGIIRGDEIARVIGELMEGEEGKRIRSKMQELKRAASAVLSKDGSSTRALEEVAKIWESKV

SEQ ID NO: 23 - DNA, 1419 bp, Rhodiola rosea
  source 1..1419: mol_type = genomic DNA
atgtcaggcacaccacacatcgccatcctccccagccccggcatgggccacctcatccccatggccgagttcgccaagcgcctagtccaccaccacaacttcagtatcaccttcgtcatccctaccgacggcccaccttcctccgcctaccaacaagtcctcacctccctcccatcttccatagatcacatcttccttccacaagtcgacttaaccgacgtcgtatcacaatcaccagctcatcccagaatcgaaaccctaatctccctcaccgtcgctcgctccctctcctccctccgcaccaccttatcctctctccaatcgtctaaaaacctcgtctcgctcgtcgttgatcttttcggcactgatgcattcgacccggccatcgagctcggcatctcgccctacattttcttcccttccacagccatgacgctctcgctcttcctatacatgcctcagcttgacaaatcagtcacgtgcgaatttcgtcacatgacggatttggttcgaattcctggatgcgttcctgtccgtggatcggatttattcgacccggttcaagacaggaccgacgaggcttataaatgggtcatacatcactccaacaggtaccctatggcggagggtgttatagagaatagcttcatggagttggaacatggtgcgttaaagtatttgcaaacggttcaatcgggtaagccgcctgtctacgcggtcggaccgttgattaaaatggattatgatgttgacgattccgggtcgaagataatcgagtggctcgatgatcaaccggttggttcggttttatttgtttcgtttggaagcggcggaacgctctcgtatgagcaaatgaccgagctggctcacggtttggaatcgagccagcaacggttcttatgggtggttcggagtccgaatcaaatccccaacagcacgtatttcagtgtacaaagccaaaaagacccgttggcttacttgccagaaggatttttaaaccgaaccgagggtaggggtctggtcgtatcgaattgggccccacaggctcaaattttgagtcacggttcgaccggtgggttcatgagccactgtggttggaattcgattttggagagtgtggtgcacggcgtgccgatcatagcgtggccgttgtacgccgagcagaagatgaattcgataatcgtggtggaggacgttaaggtggcgctgaggccggcgggggtaggggagagggtggtggagaggtcggagataaccgcagtggtgaaggcgttgatggagggtgaggaggggaagaaggtaaggaataggatgaaggaactcaaggaagcggcggcacgtgcggttagtgatgacggtgcgtcgaccatagcgattgcggacttggcgcaaaaatggcggagttcgatgaagcattga

SEQ ID NO: 24 - protein, 472 aa, Rhodiola rosea
MSGTPHIAILPSPGMGHLIPMAEFAKRLVHHHNFSITFVIPTDGPPSSAYQQVLTSLPSSIDHIFLPQVDLTDVVSQSPAHPRIETLISLTVARSLSSLRTTLSSLQSSKNLVSLVVDLFGTDAFDPAIELGISPYIFFPSTAMTLSLFLYMPQLDKSVTCEFRHMTDLVRIPGCVPVRGSDLFDPVQDRTDEAYKWVIHHSNRYPMAEGVIENSFMELEHGALKYLQTVQSGKPPVYAVGPLIKMDYDVDDSGSKIIEWLDDQPVGSVLFVSFGSGGTLSYEQMTELAHGLESSQQRFLWVVRSPNQIPNSTYFSVQSQKDPLAYLPEGFLNRTEGRGLVVSNWAPQAQILSHGSTGGFMSHCGWNSILESVVHGVPIIAWPLYAEQKMNSIIVVEDVKVALRPAGVGERVVERSEITAVVKALMEGEEGKKVRNRMKELKEAAARAVSDDGASTIAIADLAQKWRSSMKH

SEQ ID NO: 25 - DNA, 1413 bp, Rauvolfia serpentina
  source 1..1413: mol_type = genomic DNA
atggagcatacacctcacattgctatggtgcccactccgggaatgggtcatctgatccccctcgttgagttcgctaaacgactcgtcctccgtcacaactttggcgtcacttttattatcccaaccgatggacctctccctaaagcacagaagagttttcttgatgctcttcccgccggcgtaaactatgttcttcttcccccggtaagcttcgacgacttacccgctgatgttaggatagagacccgtatttgtctcaccatcactcgctctctcccgtttgttcgggatgccgttaagactctactcgccaccaccaagttagctgctctagtggtggatcttttcggcaccgatgcatttgatgttgcaattgagttcaaggtctccccttatatcttctatcctacgacggccatgtgcctgtctcttttctttcacttgcctaagcttgatcaaatggtgtcctgcgaatatagagacgtcccagaaccattgcagattccaggatgcatacccattcacgggaaggattttcttgacccagctcaggatcgcaaaaatgatgcctacaaatgcctccttcaccaggccaagagataccggttagctgagggtatcatggtcaacaccttcaacgacttggagccaggacccttaaaagctttgcaggaggaagaccagggtaagccacccgtttatccgatcggaccactcatcagagcggattcaagcagcaaggtcgacgactgtgaatgtttgaaatggctagatgaccagccacgtgggtcggttctgtttatttctttcggaagcggtggggcagtctcccataatcagttcattgagctagctttgggattagagatgagcgagcaaagattcttgtgggttgtccgaagcccaaatgataaaattgcgaatgcaacgtatttcagcattcaaaatcagaatgatgctcttgcatatctgccagaaggattcttggagagaaccaaggggcgttgtcttttggtcccgtcttgggcgccgcagactgaaattcttagccatggttccacgggtggatttctaacccactgcgggtggaactctattcttgagagtgtagttaatggggtgccgctaattgcttggcctctttatgcagagcaaaagatgaacgccgtaatgttgacggagggtcttaaagtggccctgaggccaaaagccggtgaaaatggcttgataggccgagtcgagatcgccaatgccgttaagggcttaatggagggagaggaaggaaagaagttccgcagcacaatgaaagacctaaaagatgcggcatcgagggcgctaagtgatgacggttcttcgacaaaagcactcgctgaattggcttgcaagtgggagaacaaaatttccagtacataa

SEQ ID NO: 26 - protein, 470 aa, Rauvolfia serpentina
MEHTPHIAMVPTPGMGHLIPLVEFAKRLVLRHNFGVTFIIPTDGPLPKAQKSFLDALPAGVNYVLLPPVSFDDLPADVRIETRICLTITRSLPFVRDAVKTLLATTKLAALVVDLFGTDAFDVAIEFKVSPYIFYPTTAMCLSLFFHLPKLDQMVSCEYRDVPEPLQIPGCIPIHGKDFLDPAQDRKNDAYKCLLHQAKRYRLAEGIMVNTFNDLEPGPLKALQEEDQGKPPVYPIGPLIRADSSSKVDDCECLKWLDDQPRGSVLFISFGSGGAVSHNQFIELALGLEMSEQRFLWVVRSPNDKIANATYFSIQNQNDALAYLPEGFLERTKGRCLLVPSWAPQTEILSHGSTGGFLTHCGWNSILESVVNGVPLIAWPLYAEQKMNAVMLTEGLKVALRPKAGENGLIGRVEIANAVKGLMEGEEGKKFRSTMKDLKDAASRALSDDGSSTKALAELACKWENKISST

SEQ ID NO: 27 - DNA, 1412 bp, synthetic construct
  misc_feature 1..1412: note = Consensus Sequence
  misc_difference 12..13: note = n is a, c, g, or t
  source 1..1412: mol_type = other DNA
atggaggaaacnnaccccacatcgccatcttaccaactcccggaatgggtcacctcatccctctcgttgagttygctaagcgactcgtccaccaccacaamttctcygtcacyttcatcatccctaccgatggwcctctctctaamgctcaaaaaactttccttractctctcccctcttccatagattacmtccttcttcctcctgttaacttcgacgatttacctgccgaygttaraatcgagactcgtatctcyctcaccgtcactcgttctcttccttccctycgtgatgtyttyaagtctttrgttgagacgaagaagytagttgctctcgtggttgatcttttcggcactgatgcatttgatgtkgcmattgagttcaacgtctcsccttatattttcttcccttccacygccatggcyttgtctctttttctttacttgcctaagcttgatgaaacggtgtcrtgtgagtttagrgacttgmchgaaccggttcagattccgggttgtattcctatycacgggaaggatyttctygacccggttcaggaccggaaaaacgatgcttacaaatggcttcttcatcatgcgaagaggtacagaatrgctgagggrataatrgtgaatagctttaaggaattggagccaggwgcwataaaagctttgcaagargaagaaccgggtaagccaccggtttacccggttggaccaytgattcarayggattcgggtaatraggttgacgagtcggaatgtttgaartggctggatgaccagccacgtggctcggttttatatgtttcstttggragtggtgggactctctcwcatgagcagctcattgagctwgcattrggtttggaaatgagcgagcaaagrttcttgtgggtggttagaagtccaaatgacagaatagctaatgccacktatttcaatgtacaaagtyaaaawgaccctttkgcttttttrccagaagggttcttggamaggaccaaagggagaggtttrgtggtgccgwcttgggcyccacaagctcaaattctggsccatggttcvacrggygggtttttaactcactgcggatggaactcgactctrgaaagtgtggtycacggggttccrcttatagcttggccactatatgcagagcaaaagatgaatgcrgtdatgctggcggaggatgttaaagtggcmctgaggccaaaagccggtgaaaatggatttgtgggaagagakgagattgctaragtggtgaaaggattgatggaaggtgaagaaggraaggggrtgcgsagtagaatgaaggagctgaaggatgcagctgctaaagtkcttagtgaagatggttcttccacaaaagcactagctgaattggcttmcaartggaagaaaawgacttacmaataa

SEQ ID NO: 28 - protein, 489 aa, synthetic construct
  REGION 1..489: note = Consensus Sequence
  VARIANT 2: note = misc_feature - Xaa can be any naturally occurring amino acid
  VARIANT 8..11: note = misc_feature - Xaa can be any naturally occurring amino acid
  VARIANT 40: note = misc_feature - Xaa can be any naturally occurring amino acid
  VARIANT 60: note = misc_feature - Xaa can be any naturally occurring amino acid
  VARIANT 80: note = misc_feature - Xaa can be any naturally occurring amino acid
  VARIANT 108: note = misc_feature - Xaa can be any naturally occurring amino acid
  VARIANT 114: note = misc_feature - Xaa can be any naturally occurring amino acid
  VARIANT 172: note = misc_feature - Xaa can be any naturally occurring amino acid
  VARIANT 181: note = misc_feature - Xaa can be any naturally occurring amino acid
  VARIANT 207: note = misc_feature - Xaa can be any naturally occurring amino acid
  VARIANT 211: note = misc_feature - Xaa can be any naturally occurring amino acid
  VARIANT 268: note = misc_feature - Xaa can be any naturally occurring amino acid
  VARIANT 446: note = misc_feature - Xaa can be any naturally occurring amino acid
  VARIANT 470: note = misc_feature - Xaa can be any naturally occurring amino acid
  VARIANT 476: note = misc_feature - Xaa can be any naturally occurring amino acid
  VARIANT 479..480: note = misc_feature - Xaa can be any naturally occurring amino acid
  VARIANT 482..484: note = misc_feature - Xaa can be any naturally occurring amino acid
MXETPAIXXXXQTPHIAILPSPGMGHLIPLVEFAKRLVHXHRFSVTFILPTDGPPSKAQXTFLNSLPSSIDYILLPPVNXDDLPSDVRIETRISLTVTRSLPSLRDAXKSLVEXKKLVALVVDLFGTDAFDVAIEFGVSPYIFFPSTAMALSLFLYLPKLDETVSCEFRDLXEPVRIPGCXPIHGKDLLDPVQDRKNDAYKWLLHHXKRYXMAEGIIVNSFKELEPGAIKALQEEEPGKPPVYPVGPLIQMDSGSKVEESECLKWLDXQPRGSVLYVSFGSGGTLSHEQLIELALGLEMSEQRFLWVVRSPNDGIANATYFNVQSSTDPLAFLPEGFLERTKGRGLVVPSWAPQAQILAHGSTGGFLTHCGWNSTLESVVHGVPLIAWPLYAEQKMNAVMLVEDLKVALRPKAGENGLVGREEIARVVKGLMEGEEGKGVRSRMRXLKDAAAKVLSDDGSSTKALAELAXKWKSKXVSXXQXXXHVAPQ